(19) United States
(12) Patent Application Publication
(10) Pub. No.: US 2003/0045478 A1
(43) Pub. Date: Mar. 6, 2003

(54) Gamma-ketoacid dipeptide derivatives as inhibitors of caspase-3

(76) Inventors: Yongxin Han, Kirkland (CA); Renee Aspiotis, Westmount (CA); Andre Giroux, Ste-Anne-De-Bellevue (CA); Erich L. Grimm, Baie d'Urfe (CA); Christophe Mellon, lle-Bizard (CA); Robert Zamboni, Pointe Claire (CA); Christopher I. Bayly, Beaconsfield (CA)

Correspondence Address: MERCK AND CO INC, P O BOX 2000, RAHWAY, NJ, 070650907

(21) Appl. No.: 10/017,735

(22) Filed: Dec. 14, 2001

Related U.S. Application Data
(60) Provisional application No. 60/256,022, filed on Dec. 15, 2000.

(51) Int. Cl. A61K 38/05; C07D 279/12; C07D 295/12

(52) U.S. Cl. 514/19; 540/544; 540/575; 540/609; 544/59; 544/162; 544/400; 546/229; 548/567; 562/450

(57) ABSTRACT

This invention encompasses the novel compounds of Formula I, which are useful in the treatment of caspase-3 mediated diseases.

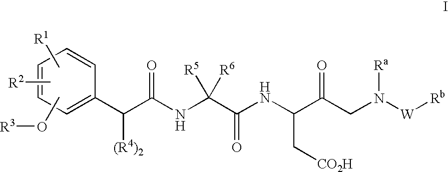

The invention also encompasses certain pharmaceutical compositions comprising compounds of Formula I as well as methods for treatment of caspase-3 mediated diseases.

### BACKGROUND OF THE INVENTION

Apoptotic cell suicide is a fundamentally important biological process that is required to maintain the integrity and homeostasis of multicellular organisms. Inappropriate apoptosis, however, underlies the etiology of many of the most intractable of human diseases. In only the last few years, many of the molecules that participate in a conserved biochemical pathway that mediates the highly ordered process of apoptotic cell suicide have been identified. At the heart of this pathway are a family of cysteine proteases, the ‘caspases’, that are related to mammalian interleukin-1β converting enzyme (ICE/caspase-1) and to CED-3, the product of a gene that is necessary for apoptotic suicide in the nematode C. elegans (Nicholson et al., 1997, Trends Biochem Sci 22:299-306). The role of these proteases in cell suicide is to disable critical homeostatic and repair processes as well as to cleave key structural components, resulting in the systematic and orderly disassembly of the dying cell.

The central importance of caspases in these processes has been demonstrated with both macromolecular and peptide-based inhibitors (which prevent apoptosis from occurring in vitro and in vivo) as well as by genetic approaches. Inhibition of apoptosis via attenuation of caspase activity should therefore be useful in the treatment of human diseases where inappropriate apoptosis is prominent or contributes to disease pathogenesis. Caspase inhibitors would thus be useful for the treatment of human diseases including, but not limited to, acute disorders such as cardiac and cerebral ischemia/reperfusion injury (e.g. stroke), spinal cord injury and organ damage during transplantation, sepsis, bacterial meningitis, as well as chronic disorders such as neurodegenerative diseases (e.g. Alzheimer's, polyglutamine-repeat disorders, Down's, spinal muscular atrophy, multiple sclerosis), immunodeficiency (e.g. HIV), diabetes, alopecia and aging.

Fourteen caspases have so far been identified in human cells. Each is synthesized as a catalytically dormant proenzyme containing an amino-terminal prodomain followed by the large and small subunits of the heterodimeric active enzyme. The subunits are excised from the proenzyme by cleavage at Asp-X junctions (Nicholson et al., 1997, Trends Biochem Sci 22:299-306). The strict requirement by caspases for Asp in the P1 position of substrates is consistent with a mechanism whereby proenzyme maturation can be either autocatalytic or performed by other caspases. The three dimensional crystal structures of mature caspase-1 and -3 show that the large subunit contains the principle components of the catalytic machinery, including the active site Cys residue which is harbored within the conserved pentapeptide motif, QACxG, and residues that stabilize the oxyanion of the tetrahedral transition state (Wilson et al., 1994, Nature 370:270-75; Walker et al., 1994, Cell 78:342-52; Rotonda et al., 1996, Nat Struct Biol 3:619-25). Both subunits contribute residues which stabilize the P1 Asp of substrates while the small subunit appears to contain most of the determinants that dictate substrate specificity and, in particular, those which form the specificity-determining S4 subsite. One distinctive feature of these proteases is the absolute requirement for an aspartic acid residue in the substrate P1 position. The carboxylate side chain of the substrate P1 Asp is tethered by four residues in caspase-1 (Arg179, Gln238 from p20 and Arg341, Ser347 from p10) that are absolutely conserved in all caspase family members. Catalysis involves a typical cysteine protease mechanism involving a catalytic dyad, composed of His237 and Cys285 (contained within an absolutely conserved QACxG pentapeptide) and an ‘oxyanion hole’ involving Gly238 and Cys285. Inhibitors bind, however, in an unexpected non-transition state configuration (which raises important considerations for inhibitor design) with the oxyanion of the thiohemiacetal being stabilized by the active site His237.

Members of the caspase family can be divided into three functional subgroups based on their substrate specificities which have been defined by a positional-scanning combinatorial substrate approach. The principle effectors of apoptosis (group II caspases, which include caspases-2,-3 and -7 as well as C. elegans CED-3) have specificity for [P4]DExD[P1], a motif found at the cleavage site of most proteins known to be cleaved during apoptosis. On the other hand, the specificity of group III caspases (caspases-6,-8,-9 and -10, as well as CTL-derived granzyme B) is [P4](I,V,L)ExD[P1] which corresponds to the activation site at the junction between the large and small subunits of other caspase proenzymes including group II (effector) family members. This and other evidence indicates that group III caspases function as upstream activators of group II caspases in a proteolytic cascade that amplifies the death signal. The role of group I caspases (caspases-1,-4 and -5) appears to be to mediate cytokine maturation and their role in apoptosis, if any, has not been substantiated.

A tetrapeptide corresponding to the substrate P4-P1 residues is sufficient for specific recognition by caspases and as a consequence has formed the basis for inhibitor design. In addition to the requirement for a P1 Asp, the P4 residue in particular appears to be most important for substrate recognition and specificity. Caspase-1, for example, prefers a hydrophobic residue such as Tyr in P4 (which corresponds to its YVHD cleavage site within proIL-1β) whereas caspase-3 (and other group II enzymes) has a preference for an anionic Asp residue (which corresponds to the DXXD cleavage sites within most polypeptides that are cleaved by these enzymes during apoptosis). Peptide aldehydes, nitrites and ketones are potent reversible inhibitors of these proteases while compounds that form thiomethylketone adducts with the active site cysteine (e.g. peptide (acyloxy)methylketones) are potent irreversible inhibitors. For example, the tetrapeptide aldehyde Ac-YVAD-CHO (which was designed to mimic the YVHD caspase-1 recognition sequence within proIL-1β) is a potent inhibitor of caspase-1 (Ki<1 nM) but a poor inhibitor of caspase-3 (Ki=12 μM) (Thornberry et al., 1992, Nature 356:768-74). In contrast, the Ac-DEVD-CHO tetrapeptide aldehyde (which was designed to mimic the caspase-3 recognition site) is a very potent inhibitor of caspase-3 (Ki<1 nM) although it is also a weaker but reasonable inhibitor of caspase-1, presumably owing to promiscuity in the S4 subsite of this enzyme (Nicholson et al., 1995, Nature 376:37-43).

Several features such as poor metabolic stability, poor membrane permeability and poor brain permeability plague these tetra-peptide derived inhibitors as a platform for drug design. The present patent application describes the resolution of some of these issues with the discovery of several novel gamma-ketoacids that make highly suitable caspase inhibitors.

### SUMMARY OF THE INVENTION

This invention encompasses the novel compounds of Formula I:

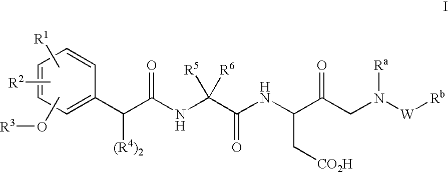

or a pharmaceutically acceptable salt, ester or hydrate thereof, wherein:

W is a bond, —CH₂—, —C(O)— or —S(O)₂—;

R^{a} is selected from R^{b} and H;

R^{b} is independently selected from the group consisting of:

(1) C₁₋₁₀alkyl or C₁₋₁₀alkoxy,

(2) C₃₋₁₁cycloalkyl or a benzofused analog thereof,

(3) phenyl or naphthyl, and

(4) HET¹, wherein HET¹ represents a 5- to 10-membered aromatic, partially aromatic or non-aromatic mono- or bicyclic ring, containing 1-3 heteroatoms selected from O, S and N,

wherein groups (1), (2) and (4) above are optionally substituted with 1-2 oxo groups, and groups (1), (2), (3) and (4) above are further optionally substituted with 1-3 substituents independently selected from the group consisting of:

(a) halo

(b) nitro,

(c) hydroxy,

(d) C₁₋₄alkyl,

(e) C₁₋₄alkoxy,

(f) C₁₋₄alkylthio,

(g) C₃₋₆cycloalkyl,

(h) phenyl or naphthyl,

(i) phenoxy, and

(j) a 5 or 6-membered aromatic or non-aromatic monocyclic ring containing 1-3 heteroatoms selected from O, S and N,

wherein groups (d)-(g) above are optionally substituted with oxo and 1-3 substituents independently selected from halo and C₁₋₄alkoxy, and groups (h)-(j) above are optionally substituted with 1-3 substituents independently selected from halo, C₁₋₄alkyl and C₁₋₄alkoxy,

or R^{a} and R^{b} may be joined together with the nitrogen atom to which they are attached to form a 3- to 10-membered non-aromatic monocyclic ring, or a benzofused analog thereof, containing 0-2 additional heteroatoms selected from O, S and N, said ring being optionally substituted with 1-2 oxo groups and further optionally substituted with 1-3 substituents independently selected from the group consisting of:

(a) halo,

(b) C₁₋₄alkyl or C₁₋₄alkoxy, each optionally substituted with 1-3 substituents independently selected from halo and C₁₋₄alkoxy, and

(c) phenyl, naphthyl or benzyl, each optionally substituted with 1-3 substituents independently selected from halo and C₁₋₄alkyl, optionally substituted with halo;

R¹ and R² are independently selected from the group consisting of:

(1) H,

(2) halo,

(3) hydroxy,

(4) nitro,

(5) cyano,

(6) C₁₋₁₀alkyl, C₃₋₁₀cycloalkyl, C₁₋₁₀alkoxy, —S(O)₀₋₂C₁₋₁₀alkyl or —NHC₁₋₁₀alkyl, each optionally substituted with 1-2 oxo groups and further optionally substituted with 1-3 substituents independently selected from the group consisting of:

(a) halo,

(b) hydroxy

(c) cyano,

(d) C₁₋₄alkoxy,

(e) —NBR⁷,

(f) phenyl or naphthyl, and

(g) HET², wherein HET² represents a 5- or 6-membered aromatic or non-aromatic monocyclic ring containing 1-4 heteroatoms selected from O, S and N, said HET² being optionally substituted with oxo and further optionally substituted with 1-2 substituents independently selected from halo and C₁₋₄alkyl, said C₁₋₄alkyl being optionally substituted with 1-3 halo groups,

(7) phenyl or naphthyl,

(8) phenoxy or —S(O)₀₋₂phenyl,

(9) —O-HET² or —S-HET², said HET² being optionally substituted with oxo and further optionally substituted as defined below, and

(10) HET³, wherein HET³ is a 5- or 6-membered aromatic or non-aromatic monocyclic ring, or a benzofused analog thereof, containing 1 to 4 heteroatoms selected from O, S and N, said HET³ being optionally substituted with oxo and further optionally substituted as defined below, wherein groups (7)-(10) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups,

or if R¹ and R² reside on adjacent atoms then R¹ and R² may be taken in combination with the carbon atoms to which they are attached to form a 5- to 7-membered aromatic or non-aromatic monocyclic ring containing 0-2 heteroatoms selected from O, S and N, said ring optionally substituted with halo and C₁₋₄alkyl, said C₁₋₄alkyl optionally substituted with halo;

R³ is C₁₋₆alkyl, optionally substituted with 1-2 oxo groups and further optionally substituted with 1-3 substituents independently selected from the group consisting of:

(a) halo,

(b) hydroxy

(c) cyano,

(d) C₁₋₄alkoxy,

(e) —NHR⁷,

(f) —S(O)₀₋₂C₁₋₄alkyl, and

(g) HET², optionally substituted with oxo and further optionally substituted with 1-2 substituents independently selected from halo or C₁₋₄alkyl, said C₁₋₄alkyl being optionally substituted with 1-3 halo groups;

each R⁴ is independently selected from the group consisting of: H, halo, hydroxy, C₁₋₆alkyl and C₁₋₄alkoxy, said C₁₋₆alkyl and C₁₋₄alkoxy being optionally substituted with 1-3 halo groups;

R⁵ is selected from the group consisting of: H, phenyl, naphthyl, C₁₋₆alkyl optionally substituted with OR⁸ and 1-3 halo groups, and C₅₋₇ cycloalkyl;

R⁶ represents H;

or R⁵ and R⁶ may be taken in combination with the carbon atom to which they are attached to form a monocyclic ring of 4-7 members, optionally containing one heteroatom selected from O, S and N, said ring optionally substituted with halo and C₁₋₄alkyl;

R⁷ is H or C₁₋₄alkyl, optionally substituted with halo; and

R⁸ is selected from the group consisting of: H, C₁₋₅alkyl optionally substituted with 1-3 halo groups, and benzyl optionally substituted with 1-3 substituents independently selected from halo, C₁₋₄alkyl and C₁₋₄alkoxy.

The invention also encompasses pharmaceutical compositions containing a compound of Formula I as well as methods for treating caspase-3 mediated diseases.

### DETAILED DESCRIPTION OF THE INVENTION

The present invention encompasses compounds represented by Formula I:

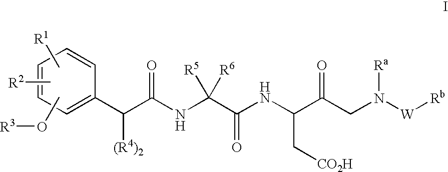

or a pharmaceutically acceptable salt, ester or hydrate thereof, wherein:

W is a bond, —CH₂—, —C(O)— or —S(O)₂—;

R^{a} is R^{b} and H;

R^{b} is independently selected from the group consisting of:

(1) C₁₋₁₀alkyl or C₁₋₁₀alkoxy,

(2) C₃-11cycloalkyl or a benzofused analog thereof,

(3) phenyl or naphthyl, and

(4) HET¹, wherein HET¹ represents a 5- to 10-membered aromatic, partially aromatic or non-aromatic mono- or bicyclic ring, containing 1-3 heteroatoms selected from O, S and N,

wherein groups (1), (2) and (4) above are optionally substituted with 1-2 oxo groups, and groups (1), (2), (3) and (4) above are further optionally substituted with 1-3 substituents independently selected from the group consisting of:

(a) halo

(b) nitro,

(c) hydroxy,

(d) C₁₋₄alkyl,

(e) C₁₋₄alkoxy,

(f) C₁₋₄alkylthio,

(g) C₃₋₆cycloalkyl,

(h) phenyl or naphthyl,

(i) phenoxy, and

(j) a 5 or 6-membered aromatic or non-aromatic monocyclic ring containing 1-3 heteroatoms selected from O, S and N, wherein groups (d)-(g) above are optionally substituted with oxo and 1-3 substituents independently selected from halo and C₁₋₄alkoxy, and groups (h)-(j) above are optionally substituted with 1-3 substituents independently selected from halo, C₁₋₄alkyl and C₁₋₄alkoxy,

or R^{a} and R^{b} may be joined together with the nitrogen atom to which they are attached to form a 3- to 10-membered non-aromatic monocyclic ring, or a benzofused analog thereof, containing 0-2 additional heteroatoms selected from O, S and N, said ring being optionally substituted with 1-2 oxo groups and further optionally substituted with 1-3 substituents independently selected from the group consisting of:

(d) halo,

(e) C₁₋₄alkyl or C₁₋₄alkoxy, each optionally substituted with 1-3 substituents independently selected from halo and C₁₋₄alkoxy, and

(f) phenyl, naphthyl or benzyl, each optionally substituted with 1-3 substituents independently selected from halo and C₁₋₄alkyl, optionally substituted with halo;

R¹ and R² are independently selected from the group consisting of:

(1) H,

(2) halo,

(3) hydroxy,

(4) nitro,

(5) cyano,

(6) C₁₋₁₀alkyl, C₃₋₁₀cycloalkyl, C₁₋₁₀alkoxy, —S(O)₀₋₂C₁₋₁₀alkyl or —NHC₁₋₁₀alkyl, each optionally substituted with 1-2 oxo groups and further optionally substituted with 1-3 substituents independently selected from the group consisting of:

(a) halo,

(b) hydroxy

(c) cyano,

(d) C₁₋₄alkoxy,

(e) —NHR⁷,

(f) phenyl or naphthyl, and

(g) HET², wherein HET² represents a 5- or 6-membered aromatic or non-aromatic monocyclic ring containing 1-4 heteroatoms selected from O, S and N, said HET² being optionally substituted with oxo and further optionally substituted with 1-2 substituents independently selected from halo and C₁₋₄alkyl, said C₁₋₄alkyl being optionally substituted with 1-3 halo groups,

(7) phenyl or naphthyl,

(8) phenoxy or —S(O)₀₋₂phenyl,

(9) —O-HET² or —S-HET², said HET² being optionally substituted with oxo and further optionally substituted as defined below, and

(10) HET³, wherein HET³ is a 5- or 6-membered aromatic or non-aromatic monocyclic ring, or a benzofused analog thereof, containing 1 to 4 heteroatoms selected from O, S and N, said HET³ being optionally substituted with oxo and further optionally substituted as defined below, wherein groups (7)-(10) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups,

or if R¹ and R² reside on adjacent atoms then R¹ and R² may be taken in combination with the carbon atoms to which they are attached to form a 5- to 7-membered aromatic or non-aromatic monocyclic ring containing 0-2 heteroatoms selected from O, S and N, said ring optionally substituted with halo and C₁₋₄alkyl, said C₁₋₄alkyl optionally substituted with halo;

R³ is C₁₋₆alkyl, optionally substituted with 1-2 oxo groups and further optionally substituted with 1-3 substituents independently selected from the group consisting of:

(a) halo,

(b) hydroxy

(c) cyano,

(d) C₁₋₄alkoxy,

(e) —NHR⁷,

(f) —S(O)₀₋₂C₁₋₄alkyl, and

(g) HET², optionally substituted with oxo and further optionally substituted with 1-2 substituents independently selected from halo or C₁₋₄alkyl, said C₁₋₄alkyl being optionally substituted with 1-3 halo groups;

each R⁴ is independently selected from the group consisting of: H, halo, hydroxy, C₁₋₆alkyl and C₁₋₄alkoxy, said C₁₋₆alkyl and C₁₋₄alkoxy being optionally substituted with 1-3 halo groups;

R⁵ is selected from the group consisting of: H, phenyl, naphthyl, C₁₋₆alkyl optionally substituted with OR⁸ and 1-3 halo groups, and C₅₋₇ cycloalkyl;

R⁶ represents H;

or R⁵ and R⁶ may be taken in combination with the carbon atom to which they are attached to form a monocyclic ring of 4-7 members, optionally containing one heteroatom selected from O, S and N, said ring optionally substituted with halo and C₁₋₄alkyl;

R⁷ is H or C₁₋₄alkyl, optionally substituted with halo; and

R⁸ is selected from the group consisting of: H, C₁₋₅alkyl optionally substituted with 1-3 halo groups, and benzyl optionally substituted with 1-3 substituents independently selected from halo, C₁₋₄alkyl and C₁₋₄alkoxy.

Another embodiment of the invention encompasses compounds of Formula I wherein R¹ is selected from the group consisting of:

(1) halo,

(2) hydroxy,

(3) nitro,

(4) cyano,

(5) C₁₋₁₀alkyl, C₃₋₁₀cycloalkyl, C₁₋₁₀alkoxy, —S(O)₀₋₂C₁₋₁₀alkyl or —NHC₁₋₁₀alkyl, each optionally substituted with 1-2 oxo groups and further optionally substituted with 1-3 substituents independently selected from the group consisting of:

(a) halo,

(b) hydroxy

(c) cyano,

(d) C₁₋₄alkoxy,

(e) —NHR⁷,

(f) phenyl or naphthyl, and

(g) HET², wherein KET² represents a 5- or 6-membered aromatic or non-aromatic monocyclic ring containing 1-4 heteroatoms selected from O, S and N, said HET² being optionally substituted with oxo and further optionally substituted with 1-2 substituents independently selected from halo and C₁₋₄alkyl, said C₁₋₄alkyl being optionally substituted with 1-3 halo groups,

(6) phenyl or naphthyl,

(7) phenoxy and —S(O)₀₋₂phenyl,

(8) —O-HET² or —S-KET², said HET² being optionally substituted with oxo and further optionally substituted as defined below, and

(9) KET³, wherein KET³ is a 5- or 6-membered aromatic or non-aromatic monocyclic ring, or a benzofused analog thereof, containing 1 to 4 heteroatoms selected from O, S and N, said KET³ being optionally substituted with oxo and further optionally substituted as defined below, wherein groups (6)-(9) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups,

or if R¹ and R² reside on adjacent atoms then R¹ and R² may be taken in combination with the carbon atoms to which they are attached to form a 5- to 7-membered aromatic or non-aromatic monocyclic ring containing 0-2 heteroatoms selected from O, S and N, said ring optionally substituted with halo and C₁₋₄alkyl, said C₁₋₄alkyl optionally substituted with halo.

Another embodiment of the invention encompasses compounds of Formula I wherein R³ is methyl, optionally substituted with 1-3 halo groups.

Another embodiment of the invention encompasses compounds of Formula I wherein R⁵ is ethyl, isopropyl or cyclopentyl and R⁶ is H.

Another embodiment of the invention encompasses compounds of Formula I wherein W is a bond.

Another embodiment of the invention encompasses compounds of Formula I wherein W is —CH₂—.

Another embodiment of the invention encompasses compounds of Formula I wherein W is —C(O)—.

Another embodiment of the invention encompasses compounds of Formula I wherein R^{a} is H or methyl, optionally substituted with 1-3 halo groups.

Within this embodiment are encompassed compounds of Formula I wherein R^{b} is phenyl or naphthyl, each optionally substituted with 1-3 substituents independently selected from the group consisting of:

(a) halo

(b) nitro,

(c) hydroxy,

(d) C₁₋₄alkyl,

(e) C₁₋₄alkoxy,

(f) C₁₋₄alkylthio, and

(g) C₃₋₆cycloalkyl,

wherein groups (d)-(g) above are optionally substituted with 1-3 substituents independently selected from halo and C₁₋₄alkoxy.

Also within this embodiment are encompassed compounds of Formula I wherein R^{b} is C₁₋₁₀alkyl or C₁₋₁₀alkoxy, each optionally substituted with 1-2 oxo groups and further optionally substituted with 1-3 substituents independently selected from the group consisting of:

(a) halo

(b) nitro,

(c) hydroxy,

(d) C₁₋₄alkoxy,

(e) C₁₋₄alkylthio,

(f) C₃₋₆cycloalkyl,

(g) phenyl or naphthyl, and

(h) phenoxy,

wherein groups (d)-(f) above are optionally substituted 1-3 substituents independently selected from halo and C₁₋₄alkoxy, and groups (g)-(h) above are optionally substituted with 1-3 substituents independently selected from halo, C₁₋₄alkyl and C₁₋₄alkoxy.

Also within this embodiment are encompassed compounds of Formula I wherein R^{b} is C₃₋₁₁cycloalkyl or a benzofused analog thereof, optionally substituted with 1-2 oxo groups and further optionally substituted with 1-3 substituents independently selected from the group consisting of:

(a) halo

(b) nitro,

(c) hydroxy,

(d) C₁₋₄alkyl,

(e) C₁₋₄alkoxy, and

(f) C₁₋₄alkylthio,

wherein groups (d)-(f) above are optionally substituted with 1-3 substituents independently selected from halo and C₁₋₄alkoxy.

Also, within this embodiment are emcompassed compounds of Formula I wherein R^{a} is HET¹, optionally substituted with 1-2 oxo groups and further optionally substituted with 1-3 substituents independently selected from the group consisting of:

(a) halo

(b) nitro,

(c) hydroxy,

(d) C₁₋₄alkyl,

(e) C₁₋₄alkoxy,

(f) C₁₋₄alkylthio, and

(g) C₃₋₆cycloalkyl,

wherein groups (d)-(g) above are optionally substituted with 1-3 substituents independently selected from halo and Cl-4alkoxy.

Also within this embodiment are encompassed compounds of Formula I wherein HET¹ represents a member selected from the group consisting of: pyridine, pyrimidin, pyridazine, pyrazine, furan, thiophene, thiazole, oxazole and isooxazole, or a benzofused or hydrogenated analog thereof, or both, each optionally substituted with 1-2 oxo groups and further optionally substituted with 1-3 substituents independently selected from the group consisting of:

(a) halo

(b) nitro,

(c) hydroxy,

(d) C₁₋₄alkyl,

(e) C₁₋₄alkoxy,

(f) C₁₋₄alkylthio, and

(g), C₃₋₆cycloalkyl,

wherein groups (d)-(g) above are optionally substituted with 1-3 substituents independently selected from halo and C₁₋₄alkoxy, or

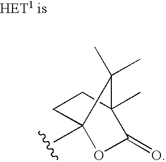

Another embodiment of the invention encompasses compounds of Formula I wherein HET² is selected from the group consisting of: butyrolactone, tetrahydrofuran, tetrahydropyran, 2-pyrrolidinone, pyridine and pyrimidine, each optionally substituted with 1-2 substituents independently selected from halo or C₁₋₄alkyl, said C₁₋₄alkyl being optionally substituted with 1-3 halo groups.

Another embodiment of the invention encompasses compounds of Formula I wherein HET³ is selected from the group consisting of: 1,2,3-oxadiazole, 1,2,4-oxadiazole, 1,3,4-oxadiazole, 1,2,3-thiadiazole, 1,2,4-thiadiazole, 1,3,4-thiadiazole, thiophene, pyrrole, pyridine, tetrazole, oxazole, thiazole, 1,2,3-triazole, 1,2,4-triazole and 1,3,4-triazole, each optionally substituted with 1-2 substituents independently selected from halo or C₁₋₄alkyl, said C₁₋₄alkyl being optionally substituted with 1-3 halo groups.

Another embodiment of the invention encompasses compounds of Formula I wherein R^{a} and R^{b} are joined with the nitrogen atom to which they are attached to form a 3- to 6-membered non-aromatic monocyclic ring, or a benzofused analog thereof, containing 0-2 additional heteroatoms selected from O, S and N, said ring being optionally substituted with 1-2 oxo groups and further optionally substituted with 1-3 substituents independently selected from the group consisting of:

(a) halo,

(b) C₁₋₄alkyl or C₁₋₄alkoxy, each optionally substituted with 1-3 substituents independently selected from halo and C₁₋₄alkoxy, and

(c) phenyl, naphthyl or benzyl, each optionally substituted with 1-3 substituents independently selected from halo and C₁₋₄alkyl, optionally substituted with halo.

Another embodiment of the invention encompasses compounds of Formula I:

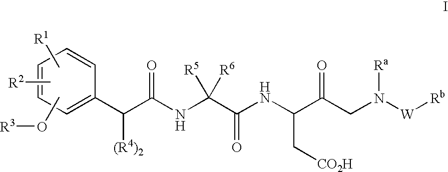

or a pharmaceutically acceptable salt, ester or hydrate thereof, wherein:

W is a bond, —CH₂—, —C(O)— or —S(O)₂—;

R^{a} is H or methyl, optionally substituted with 1-3 halo groups;

R^{b} is independently selected from the group consisting of:

(1) C₁₋₁₀alkyl or C₁₋₁₀alkoxy,

(2) C₃₋₁₁cycloalkyl or a benzofused analog thereof,

(3) phenyl or naphthyl, and

(4) HET¹, wherein HET¹ represents a 5- to 10-membered aromatic, partially aromatic or non-aromatic mono- or bicyclic ring, containing 1-3 heteroatoms selected from O, S and N,

wherein groups (1), (2) and (4) above are optionally substituted with 1-2 oxo groups, and groups (1), (2), (3) and (4) above are further optionally substituted with 1-3 substituents independently selected from the group consisting of:

(a) halo

(b) nitro,

(c) hydroxy,

(d) C₁₋₄alkyl,

(e) C₁₋₄alkoxy,

(f) C₁₋₄alkylthio,

(g) C₃₋₆cycloalkyl,

(h) phenyl or naphthyl,

(i) phenoxy, and

(5) a 5 or 6-membered aromatic or non-aromatic monocyclic ring containing 1-3 heteroatoms selected from O, S and N, wherein groups (d)-(g) above are optionally substituted with oxo and 1-3 substituents independently selected from halo and C₁₋₄alkoxy, and groups (h)-(O) above are optionally substituted with 1-3 substituents independently selected from halo, C₁₋₄alkyl and C₁₋₄alkoxy,

or R^{a} and R^{b} may be joined with the nitrogen atom to which they are attached to form a 3- to 6-membered non-aromatic monocyclic ring, or a benzofused analog thereof, containing 0-2 additional heteroatoms selected from O, S and N, said ring being optionally substituted with 1-2 oxo groups and further optionally substituted with 1-3 substituents independently selected from the group consisting of:

(a) halo,

(b) C₁₋₄alkyl or C₁₋₄alkoxy, each optionally substituted with 1-3 substituents independently selected from halo and C₁₋₄alkoxy, and

(c) phenyl, naphthyl or benzyl, each optionally substituted with 1-3 substituents independently selected from halo and C₁₋₄alkyl optionally substituted with halo;

R¹ is selected from the group consisting of:

(1) halo,

(2) methoxy,

(3) acetyl, and

(4) 1,2,4-oxadiazol-5-yl, optionally substituted at the 3-position with methyl;

R² is H;

R³ is methyl, optionally substituted with 1-3 halo groups;

each R⁴ is independently selected from the group consisting of: H and hydroxy;

R⁵ is selected from the group consisting of: H, C₁₋₆alkyl optionally substituted with 1-3 halo groups, and C₅₋₇ cycloalkyl; and

R⁶ represents H.

Within this embodiment are encompassed compounds wherein R⁵ is ethyl, isopropyl or cyclopentyl.

Within this embodiment are encompassed compounds wherein: HET¹ is selected from the group consisting of: pyridine, pyrimidine, pyridazine, pyrazine, furan, thiophene, thiazole, oxazole and isooxazole, or a benzofused or hydrogenated analog thereof, or both, each optionally substituted with 1-2 oxo groups and further optionally substituted with 1-3 substituents independently selected from the group consisting of:

(a) halo

(b) nitro,

(c) hydroxy,

(d) C₁₋₄alkyl,

(e) C₁₋₄alkoxy,

(f) C₁₋₄alkylthio, and

(g) C₃₋₆cycloalkyl,

wherein groups (d) to (g) above are optionally substituted with 1-3 substituents independently selected from halo and C₁₋₄alkoxy, or

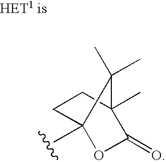

For purposes of this specification alkyl means linear or branched structures and combinations thereof, containing one to twenty carbon atoms unless otherwise specified. Examples of alkyl groups include methyl, ethyl, propyl, isopropyl, butyl, s- and t-butyl, pentyl, hexyl, heptyl, octyl, nonyl, undecyl, dodecyl, tridecyl, tetradecyl, pentadecyl, eicosyl, 3,7-diethyl-2,2-dimethyl-4-propylnonyl, and the like.

Cycloalkyl means mono- , bi- or tri-cyclic structures, optionally combined with linear or branched structures, containing one to twenty carbon atoms unless otherwise specified. Examples of cycloalkyl groups include cyclopropyl, cyclopentyl, cycloheptyl, adamantly, cyclododecylmethyl, 2-ethyl-1-bicyclo[4.4.0]decyl, and the like.

Alkoxy means alkoxy groups of one to ten carbon atoms, unless otherwise specified, of a straight, branched or cyclic configuration. Examples of alkoxy groups include methoxy, ethoxy, propoxy, isopropoxy, and the like.

Alkylthio means alkylthio groups of one to ten carbon atoms, unless otherwise specified, of a straight, branched or cyclic configuration. Examples of alkylthio groups include methylthio, propylthio, isopropylthio, etc. By way of illustration, the propylthio group signifies —SCH₂CH₂CH₃.

Halo includes F, Cl, Br and I.

HET¹ is defined as a 5- to 10-membered aromatic, partially aromatic or non-aromatic mono- or bicyclic ring, containing 1-3 heteroatoms selected from O, S and N. Preferably, HET¹ is a 5- or 6-membered aromatic or non-aromatic monocyclic ring containing 1-3 heteroatoms selected from O, S and N, for example, pyridine, pyrimidin, pyridazine, furan, thiophene, thiazole, oxazole, isooxazole and the like, or HET¹ is a 9- or 10-membered aromatic or partially aromatic bicyclic ring containing 1-3 heteroatoms selected from O, S, and N, for example, benzofuran, benzothiophene, indole, pyranopyrrole, benzopyran, quionoline, benzocyclohexyl, naphtyridine and the like.

HET² is defined as a 5- or 6-membered aromatic or non-aromatic monocyclic ring containing 1-4 heteroatoms selected from O, S and N. Examples of HET² include butyrolactone, tetrahydrofuran, tetrahydropyran, 2-pyrrolidinone, pyridine, pyrimidine and oxatriazole.

HET³ is defined as a 5- or 6-membered aromatic or non-aromatic monocyclic ring, or a benzofused analog thereof, containing 1 to 4 heteroatoms selected from O, S and N. Examples of HET³ include 1,2,3-oxadiazole, 1,2,4-oxadiazole, 1,3,4-oxadiazole, 1,2,3-thiadiazole, 1,2,4-thiadiazole, 1,3,4-thiadiazole, thiophene, pyrrole, pyridine, tetrazole, oxazole, thiazole, 1,2,3-triazole, 1,2,4-triazole, 1,3,4-triazole, 1,2,3,4 oxatriazole. Examples of HET³ as a benzofused analog include benzothiophene, indole, indazole, benzoxazole and benzopyran.

For purposes of this specification, the following abbreviations have the indicated meanings:

| | | |
|---|---|---|
| AcOH | = | acetic acid |
| Alloc | = | allyloxycarbonyl |
| APCI | = | atmospheric pressure chemical ionization |
| BOC | = | t-butyloxycarbonyl |
| CBZ | = | carbobenzoxy |
| DCC | = | 1,3-dicyclohexylcarbodiimide |
| DIBAL | = | diisobutyl aluminum hydride |
| DIEA | = | N,N-diisoproylethylamine |
| DMAP | = | 4-(dimethylamino)pyridine |
| DMF | = | dimethyl formamide |
| DTT | = | dithiothreitol |
| EDCI | = | 1-(3-dimethylaminopropyl)-3-ethylcarbodiimide |
| | | hydrochloride |
| EDTA | = | ethylenediaminetetraacetic acid, tetrasodium salt |
| | | hydrate |
| ESI | = | electrospray ionization |
| FAB | = | fast atom bombardment |
| FMOC | = | 9-fluorenylmethoxycarbonyl |
| HMPA | = | hexamethylphosphoramide |
| HATU | = | O-(7-Azabenzotriazol-l-yl)N,N,N′,N′- |
| | | tetramethyluronium hexafluorophosphate |
| HOBT | = | 1-hydroxybenzotriazole |
| HRMS | = | high resolution mass spectrometry |
| ICl | = | iodine monochloride |
| IBCF | = | isobutyl chloroformate |
| KHMDS | = | potassium hexamethyldisilazane |
| LDA | = | lithium diisopropylamide |
| MCPBA | = | metachloroperbenzoic acid |
| Ms | = | methanesulfonyl = mesyl |
| MsO | = | methanesulfonate = mesylate |
| NBS | = | N-bromosuccinimide |
| NMM | = | 4-methylmorpholine |
| PCC | = | pyridinium chlorochromate |
| PDC | = | pyridinium dichromate |
| Ph | = | phenyl |
| PPTS | = | pyridinium p-toluene sulfonate |
| pTSA | = | p-toluene sulfonic acid |
| r.t. | = | room temperature |
| rac. | = | racemic |
| TFA | = | trifluoroacetate |
| TfO | = | trifluoromethanesulfonate = triflate |
| TLC | = | thin layer chromatography |

Alkyl group abbreviations:

| | | |
|---|---|---|
| Me | = | methyl |
| Et | = | ethyl |
| n-Pr | = | normal propyl |
| i-Pr | = | isopropyl |
| n-Bu | = | normal butyl |
| i-Bu | = | isobutyl |
| s-Bu | = | secondary butyl |
| t-Bu | = | tertiary butyl |

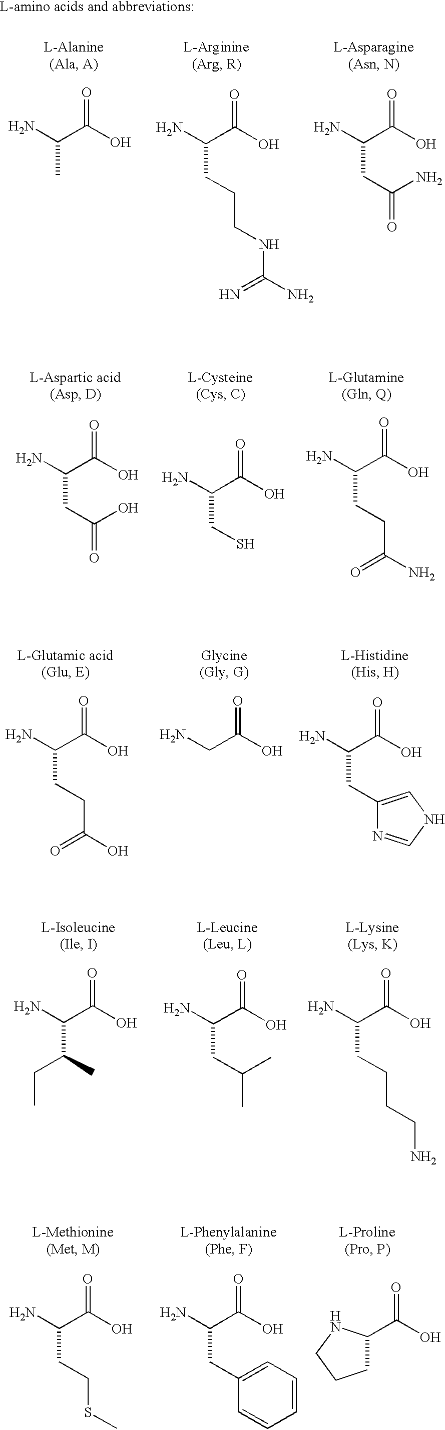

Representative examples of compounds of Formula I are found in Table I below.

| Example # | Structure | m/z |
|---|---|---|
| 1 | 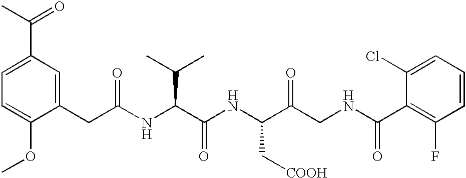 | −APCI: 590.2 (M − 1)⁻ |
| 2 | 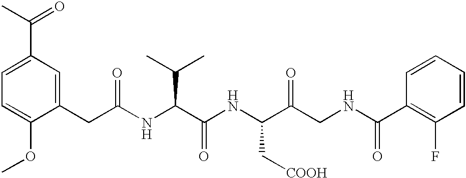 | −APCI: 556.5 (M − 1)⁻ |
| 3 | 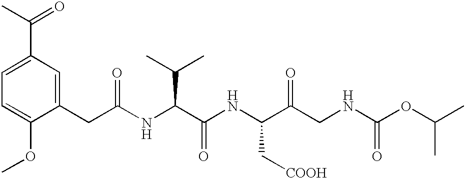 | −APCI: 520.4 (M + 1)⁻ |
| 4 | 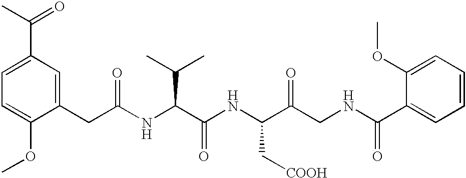 | −APCI: 568.6 (M − 1)⁻ |
| 5 | 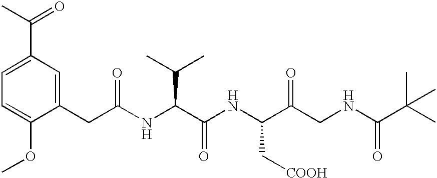 | −APCI: 518.4 (M − 1)⁻ |
| 6 | 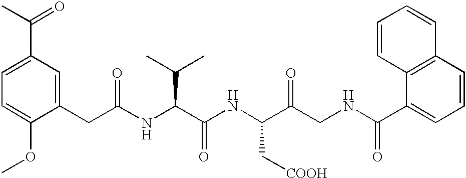 | −APCI: 588.3 (M − 1)⁻ |
| 7 | 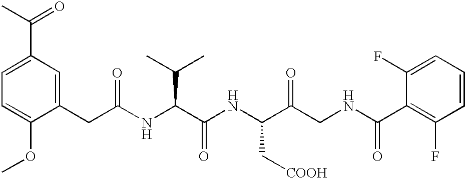 | −APCI: 574.4 (M − 1)⁻ |
| 8 | 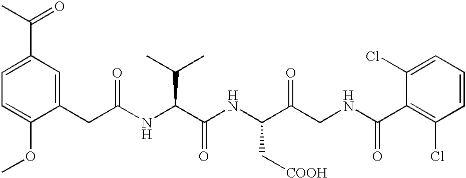 | −APCI: 606.4 (M − 1)⁻ |
| 9 | 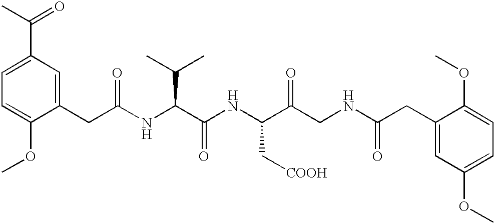 | −APCI: 612.6 (M − 1)⁻ |
| 10 | 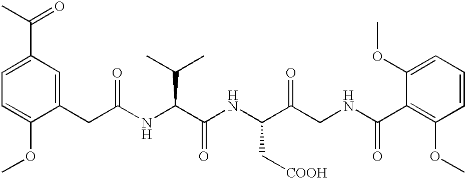 | −APCI: 598.5 (M − 1)⁻ |
| 11 | 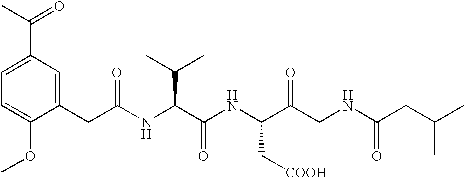 | −APCI: 518.4 (M − 1)⁻ |
| 12 | 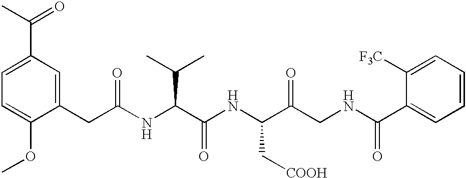 | −APCI: 606.5 (M − 1)⁻ |
| 13 | 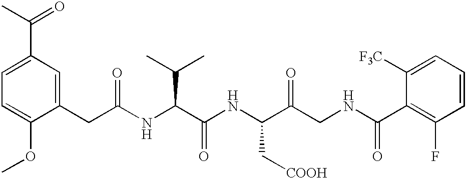 | −APCI: 624.5 (M − 1)⁻ |
| 14 | 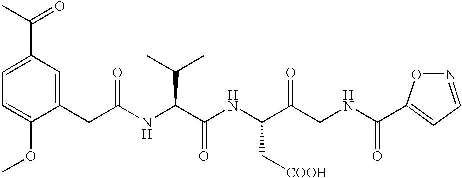 | −APCI: 529.3 (M − 1)⁻ |
| 15 | 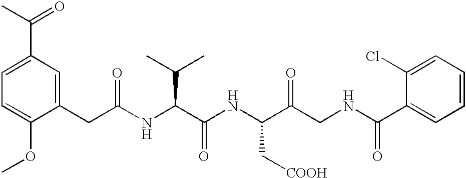 | −APCI: 572.8 (M − 1)⁻ |
| 16 | 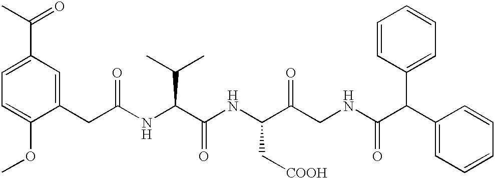 | −APCI: 628.3 (M − 1)⁻ |
| 17 | 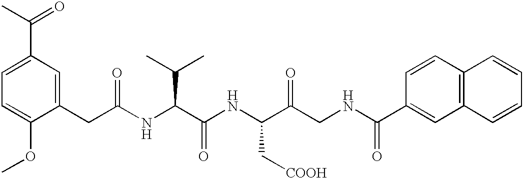 | −APCI: 588.3 (M − 1)⁻ |
| 18 | 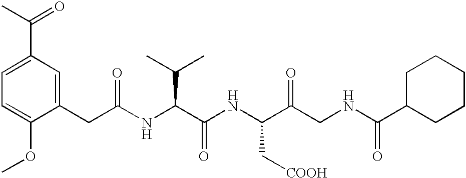 | −APCI: 544.5 (M − 1)⁻ |
| 19 | 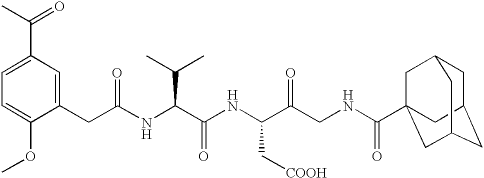 | −APCI: 598.0 (M − 1)⁻ |
| 20 | 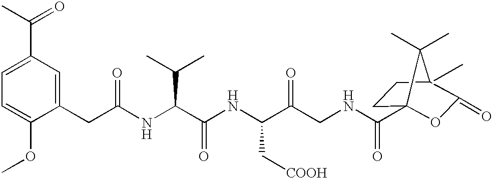 | −APCI: 615.9 (M − 1)⁻ |
| 21 | 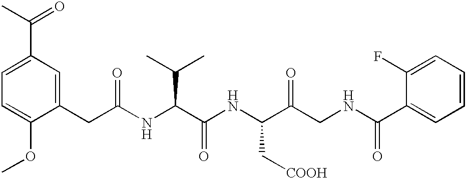 | −APCI: 592.5 (M − 1)⁻ |
| 22 | 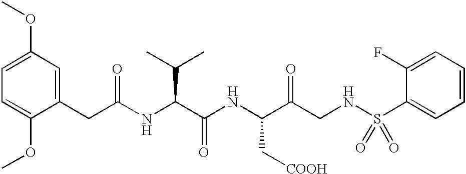 | −APCI: 580.6 (M − 1)⁻ |
| 23 | 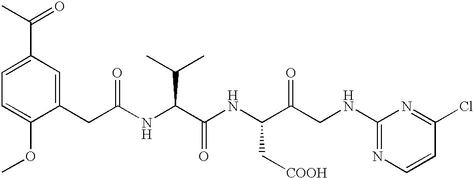 | −APCI: 546.4 (M − 1)⁻ |
| 24 | 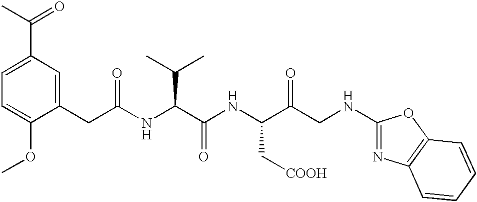 | −APCI: 551.4 (M − 1)⁻ |
| 25 | 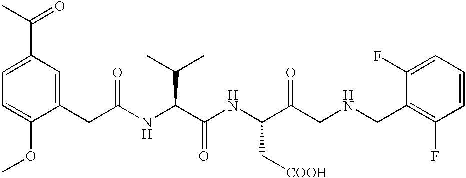 | +ESI: 563.3 (M + 1)⁺ |
| 26 | 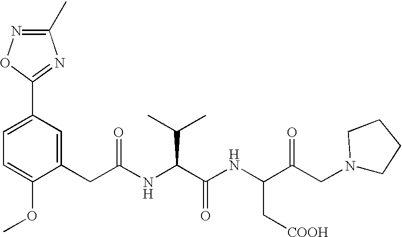 | +ESI: 531.2 (M + 1)⁺ |
| 27 | 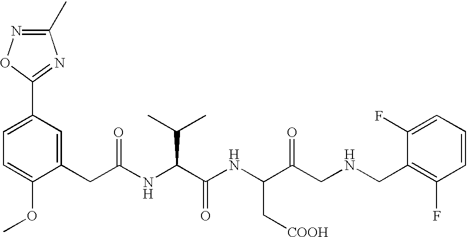 | −APCI: 600.8 (M − 1)⁻ |
| 28 | 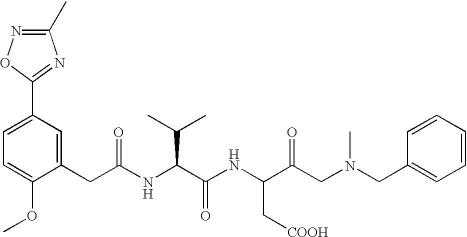 | +ESI: 580.1 (M + 1)⁺ |
| 29 | 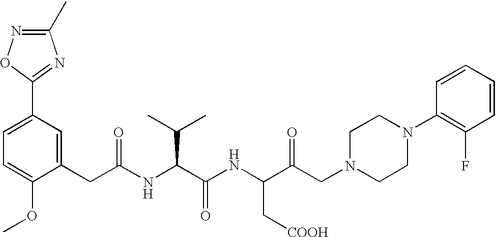 | +APCI: 639.4 (M + 1)⁺ |
| 30 | 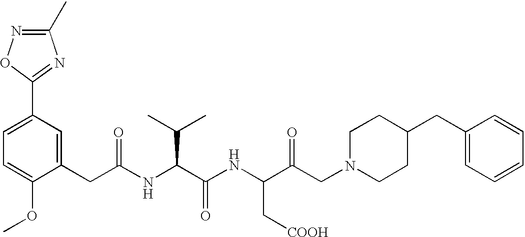 | −APCI: 632.5 (M − 1)⁻ |
| 31 | 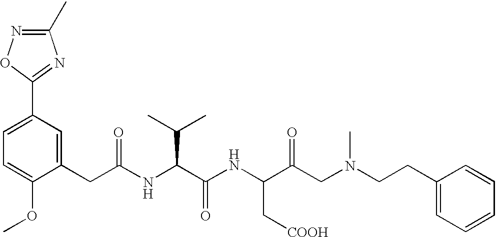 | +ESI: 591.1 (M + 1)⁺ |
| 32 | 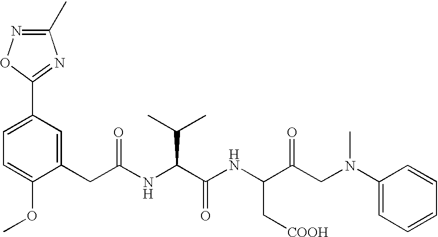 | −APCI: 564.6 (M − 1)⁻ |
| 33 | 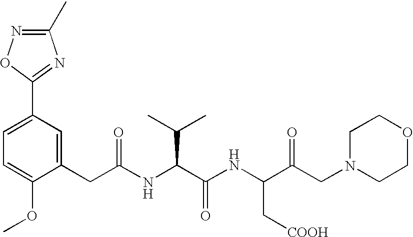 | +ESI: 545.9 (M + 1)⁺ |
| 34 | 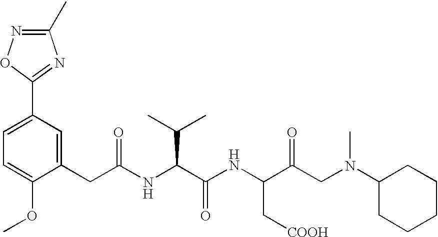 | +ESI: 573.0 (M + 1)⁺ |
| 35 | 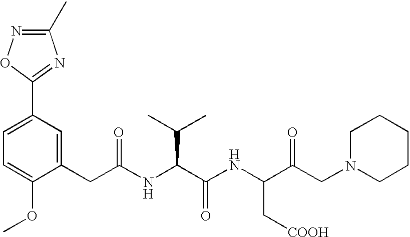 | +ESI: 544.0 (M + 1)⁺ |
| 36 | 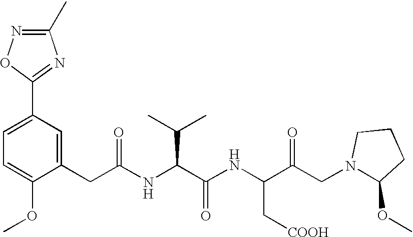 | −APCI: 572.4 (M − 1)⁻ |
| 37 | 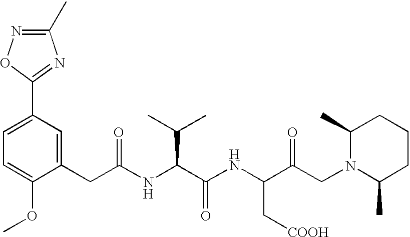 | −APCI: 570.9 (M − 1)⁻ |
| 38 | 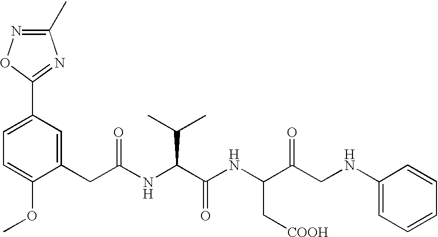 | +ESI: 552.1 (M + 1)⁺ |
| 39 | 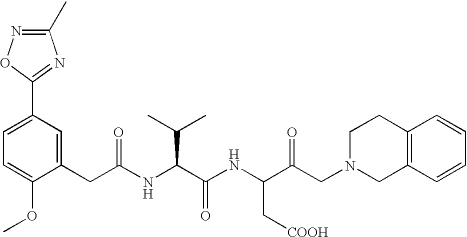 | +APCI: 592.5 (M + 1)⁺ |
| 40 | 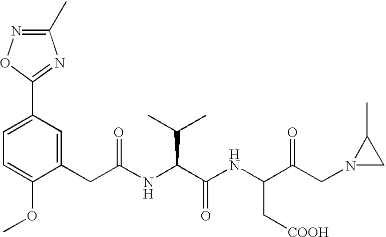 | +ESI: 516.1 (M + 1)⁺ |
| 41 | 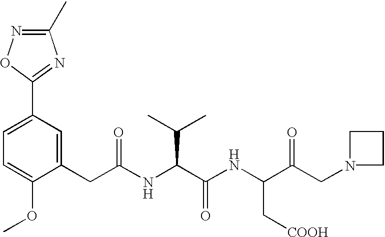 | +ESI: 516.1 (M + 1)⁺ |
| 42 | 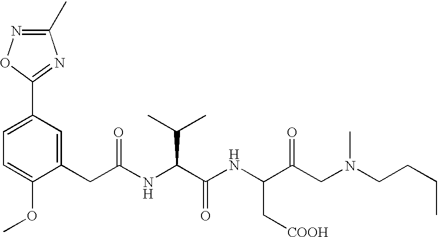 | +ESI: 548.1 (M + 1)⁺ |
| 43 | 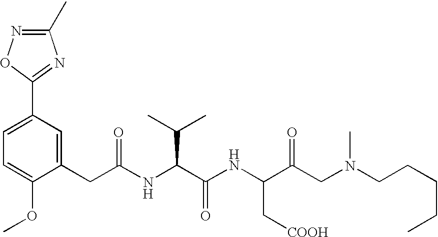 | |
| 44 | 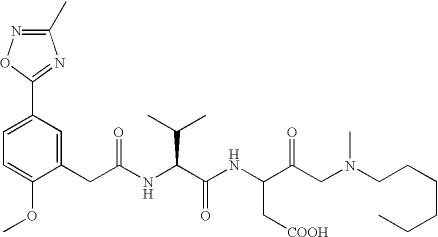 | |
| 45 | 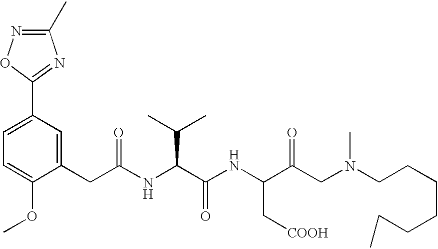 | |
| 46 | 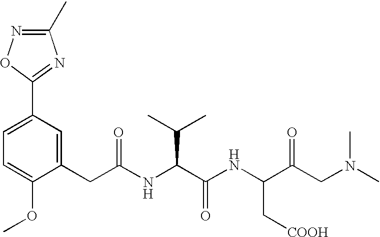 | |
| 47 | 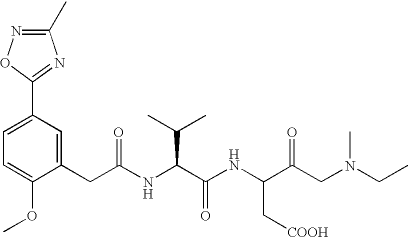 | |
| 48 | 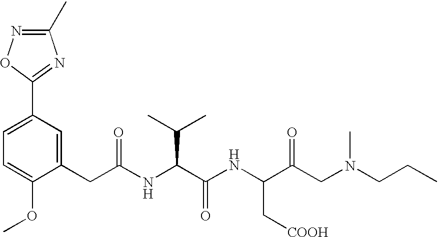 | |
| 49 | 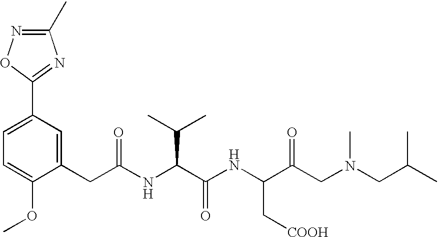 | |
| 50 | 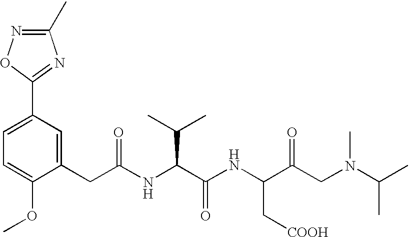 | |
| 51 | 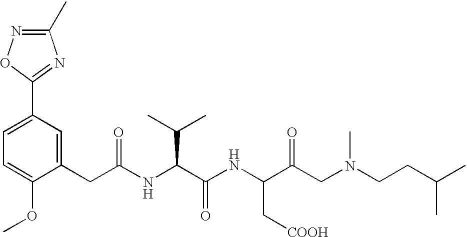 | |
| 52 | 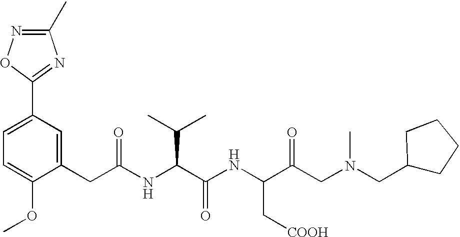 | |
| 53 | 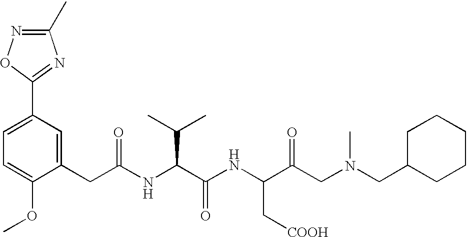 | |
| 54 | 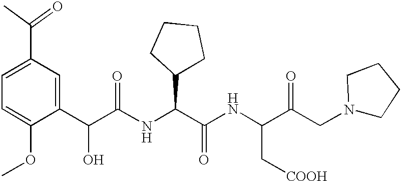 | +ESI: 532 (M + 1)⁺ |
| 55 | 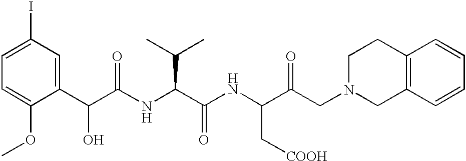 | +APCI: 652 (M + 1)⁺ |
| 56 | 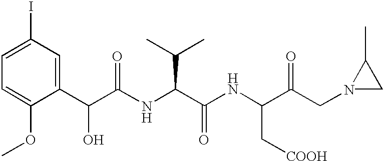 | +APCI: 576 (M + 1)⁺ |
| 57 | 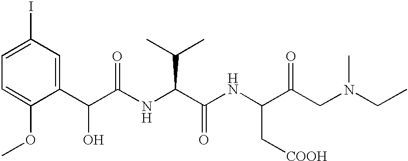 | +APCI: 578 (M + 1)⁺ |
| 58 | 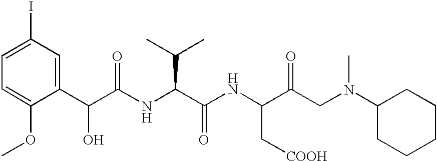 | +APCI: 632 (M + 1)⁺ |
| 59 | 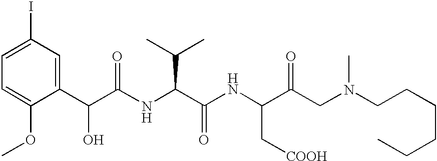 | +APCI: 635 (M + 1)⁺ |
| 60 | 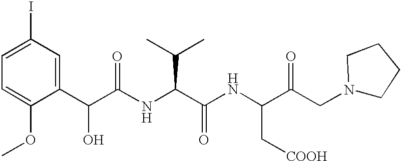 | +APCI: 590 (M + 1)⁺ |
| 61 | 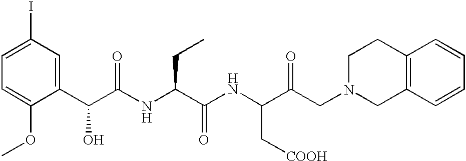 | +APCI: 638.3 (M + 1)⁺ |
| 62 | 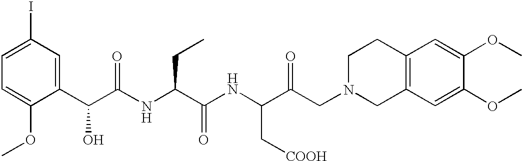 | +APCI: 698.3 (M + 1)⁺ |
| 63 | 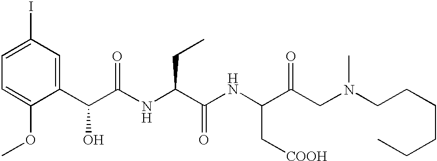 | +APCI: 620.4 (M + 1)⁺ |

The compounds described herein, and in particular, in Table I, are intended to include salts, enantiomers, esters and hydrates, in pure form and as a mixture thereof. Also, when a nitrogen atom appears, it is understood sufficient hydrogen atoms are present to satisfy the valency of the nitrogen atom.

While chiral structures are shown below, by substituting into the synthesis schemes an enantiomer other than the one shown, or by substituting into the schemes a mixture of enantiomers, a different isomer or a racemic mixture can be achieved. Thus, all such isomers and mixtures are included in the present invention.

The compounds described typically contain asymmetric centers and may thus give rise to diastereomers and optical isomers. The present invention is meant to comprehend such possible diastereomers as well as their racemic and resolved, enantiomerically pure forms and pharmaceutically acceptable salts thereof.

Some of the compounds described herein contain olefinic double bonds, and unless specified otherwise, are meant to include both E and Z geometric isomers.

This invention also encompasses a pharmaceutical composition comprised of a compound of Formula I in combination with a pharmaceutically acceptable carrier.

This invention also encompasses a method of treating or preventing a caspase-3 mediated disease or condition in a mammalian patient in need of such treatment, comprising administering to said patient a compound of Formula I in an amount effective to treat or prevent said caspase-3 mediated disease.

Another embodiment of the invention encompasses the method of treating or preventing a caspase-3 mediated disease wherein the disease or condition is selected from the group consisting of: cardiac or cerebral ischemia or reperfusion F; injury, type I diabetes, immune deficiency syndrome or AIDS, cerebral or spinal cord trauma injury, organ damage during transplantation, alopecia, aging, Parkinson's disease, Alzheimer's disease, Down's syndrome, spinal muscular atrophy, multiple sclerosis, neurodegenerative disorders, sepsis and bacterial meningitis.

Another embodiment of the invention encompasses the method of treating or preventing a caspase-3 mediated disease wherein the disease or condition is cardiac and cerebral ischemia or reperfusion injury, spinal cord injury and organ damage during transplantation.

Another embodiment of the invention encompasses the method of treating or preventing a caspase-3 mediated disease wherein the disease or condition is a chronic disorder selected from the group consisting of: a neurodegenerative disease selected from Alzheimer's, polyglutamine-repeat disorders, Down's syndrome, spinal muscular atrophy, multiple sclerosis, immunodeficiency, HIV, diabetes, alopecia and aging.

Another embodiment of the invention encompasses the method of treating or preventing a caspase-3 mediated disease wherein the disease or condition is cardiac or cerebral ischemia or reperfusion injury. Another embodiment of the invention encompasses the method of treating or preventing a caspase-3 mediated disease wherein the disease or condition is bacterial meningitis. Another embodiment of the invention encompasses the method of treating or preventing a caspase-3 mediated disease wherein the disease or condition is sepsis.

The pharmaceutical compositions of the present invention comprise a compound of Formula I as an active ingredient or a pharmaceutically acceptable salt thereof in combination with a pharmaceutically acceptable carrier, and optionally other therapeutic ingredients. The term “pharmaceutically acceptable salts” refers to salts prepared from pharmaceutically acceptable bases including inorganic bases and organic bases. Representative salts derived from inorganic bases include aluminum, ammonium, calcium, copper, ferric, ferrous, lithium, magnesium, manganic salts, manganous, ammonium, potassium, sodium, zinc and the like. Particularly preferred are the calcium, magnesium, potassium, and sodium salts. Representative salts derived from pharmaceutically acceptable organic bases include salts of primary, secondary and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, salts may be prepared from pharmaceutically acceptable non-toxic acids, including inorganic and organic acids. Examples of such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric and tartaric acids.

In the discussion of methods of treatment that follows, reference to the compounds of Formula I are meant to also include the pharmaceutically acceptable salts.

The ability of the compounds of Formula I to inhibit caspase-3 make them useful research tools in the field of apoptosis.

The magnitude of therapeutic dose of a compound of Formula I will, of course, vary with the nature of the severity of the condition to be treated and with the particular compound of Formula I and its route of administration and vary upon the clinician's judgement. It will also vary according to the age, weight and response of the individual patient. An effective dosage amount of the active component can thus be determined by the clinician after a consideration of all the criteria and using is best judgement on the patient's behalf. A representative dose will range from 0.001 mpk/d to about 100 mpk/d.

An ophthalmic preparations for ocular administration comprising 0.001-1% by weight solutions or suspensions of the compounds of Formula I in an acceptable ophthalmic formulation may be used.

Any suitable route of administration may be employed for providing an effective dosage of a compound of the present invention. For example, oral, parenteral and topical may be employed. Dosage forms include tablets, troches, dispersions, suspensions, solutions, capsules, creams, ointments, aerosols, and the like.

The compositions include compositions suitable for oral, parenteral and ocular (ophthalmic). They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration. In preparing the compositions for oral dosage form, any of the usual pharmaceutical media may be employed, such as, for example, water, alcohols, oils, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case or oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

Pharmaceutical compositions of the present invention suitable for oral administration may be presented as discrete units such as capsules, cachets or tablets each containing a predetermined amound of the active ingredient, as a powder or granules or as a solution or a suspension in an aqueous liquid, a non-aqueous liquid, an oil-in-water emulsion or a water-in-oil emulsion. Such compositions may be prepared by any of the methods of pharmacy but all methods include the step of bringing into active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. For example, each dosage unit may contain from about 0.01 mg to about 1.0 g of the active ingredient.

### Methods of Synthesis

The compounds of the present invention are prepared using the general procedures described below:

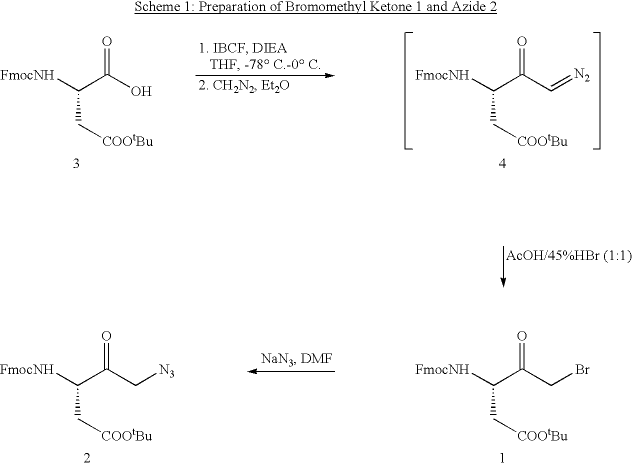

Bromomethyl ketone 1 is prepared as illustrated in Scheme 1. Reaction of N-fluorenylmethyloxycarbonyl-L-aspartic acid β-tert-butyl ester (Fmoc-L-Asp (OtBu)-OH) (3) (Novabiochem) with iso-butyl chloroformate (IBCF) followed by treating the reaction mixture with an excess of diazomethane yields the diazomethyl ketone intermediate 4. This intermediate is subjected in situ to a 1:1 mixture of AcOH and 45% aqueous hydrobromic acid (HBr) to give compound 1 as a white powder. Treatment of bromide 1 with sodium azide in dimethyl formamide (DMF) affords azide 2.

The semicarbazide Resin A is prepared according to Scheme 2. Treatment of compound 5 (Webb et al, J. Am. Chem. Soc. 114, 3156 (1992)) with a commercial amino-Merrifield resin in the presence of 1-(3-dimethylaminopropyl)-3-ethylcarbodiimide hydrochloride (EDCI) and 1-hydroxybenzotriazole (HOBT) in dichloromethane followed by removal of the Boc group with trifluoroacetic acid (TFA) in dichloromethane furnishes Resin A.

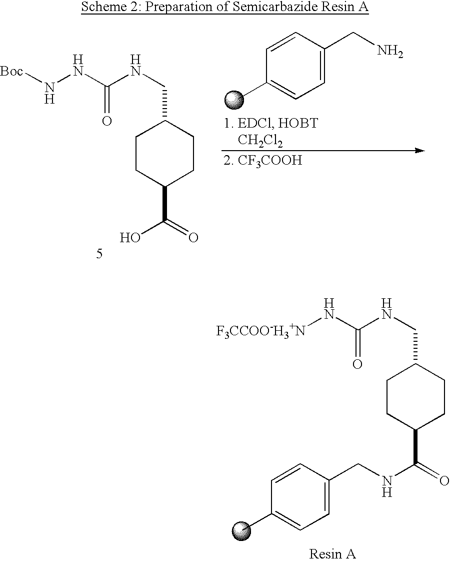

The general procedure for solid phase synthesis of compound of general structure I incorporating either an amide P1′ side chain or a P1′ sulfonamide side chain is illustrated in Scheme 3. Ketone 2 is reacted with Resin A in TIF in the presence of acetic acid to afford Resin B. The Fmoc group in Resin B is removed with 20% (v) piperidine (Pip) in DMF and the resultant resin reacted with FmocHNCR⁵(R⁶)COOH using O-(7-Azabenzotriazol-1-yl)N,N,N′,N′-tetramethyluronium hexafluorophosphate (HATU) as the activating agent and diisopropylethylamine (DIEA) as the base, affording Resin C. The Fmoc group in Resin C is cleaved similarly and then the amino group released is reacted with acid 6 as shown to yield Resin D. The azide functionality in Resin D is reduced to the NH₂ group using dithiothreitol (DTT) and DIEA in DMF as described in the literature (see: Meldel, M. et al., Tetrahedron Lett. 38, 2531 (1997)). The amino group is reacted with an acid chloride (RbWX where W=CO and X=Cl) in the presence of DEA in dichloromethane, or a carboxylic acid (RbWX where W=CO and X=OH) in the presence of a coupling reagent such as HATU or EDCI and a suitable base, or a suitable sulfonyl chloride (RbWX where W=SO₂ and X=Cl) in the presence of DIEA in dichloromethane. The resin thus obtain is treated with a solution of TFA/H₂O(9/1 v/v) to give compounds of general structure I.

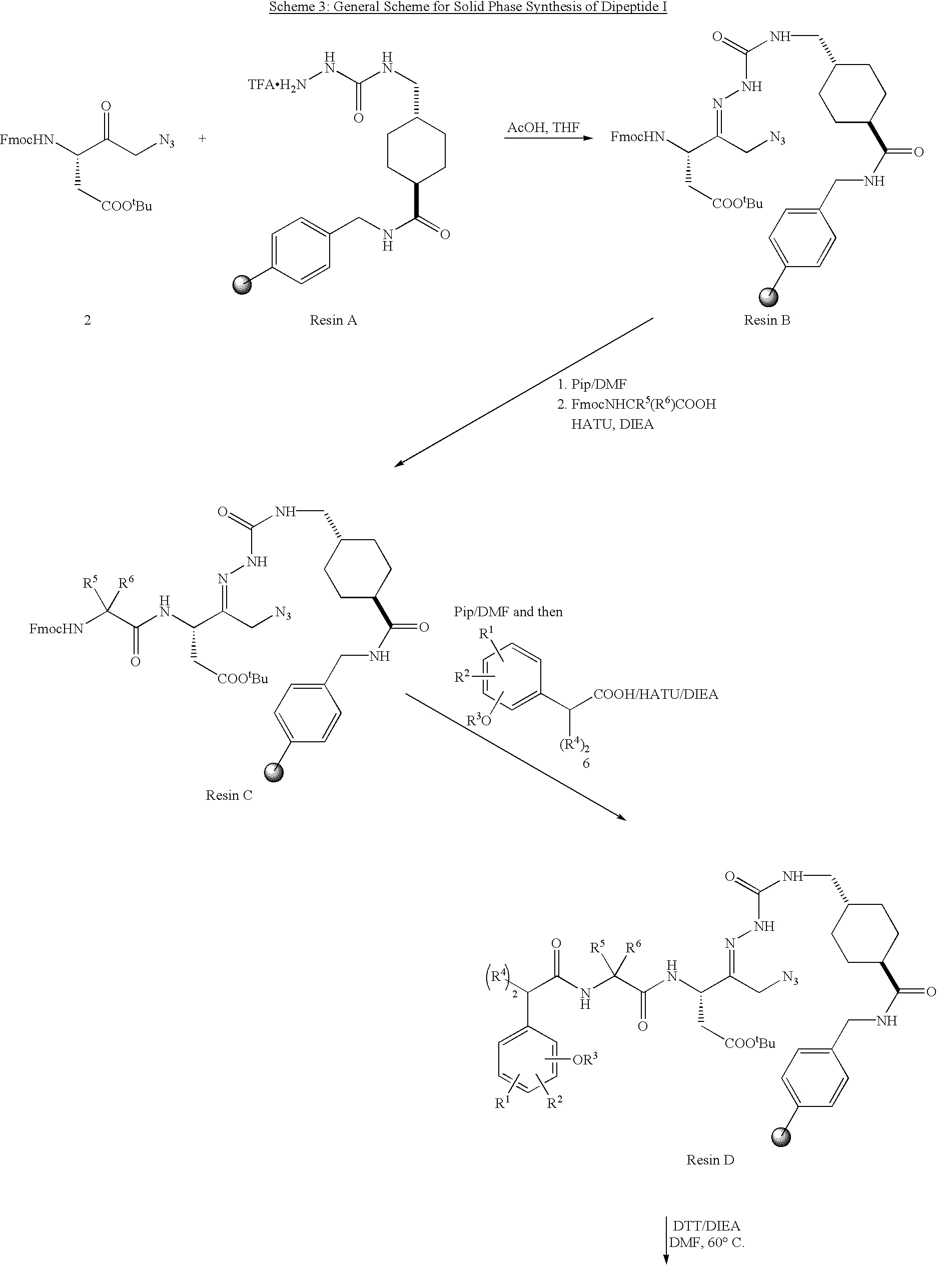

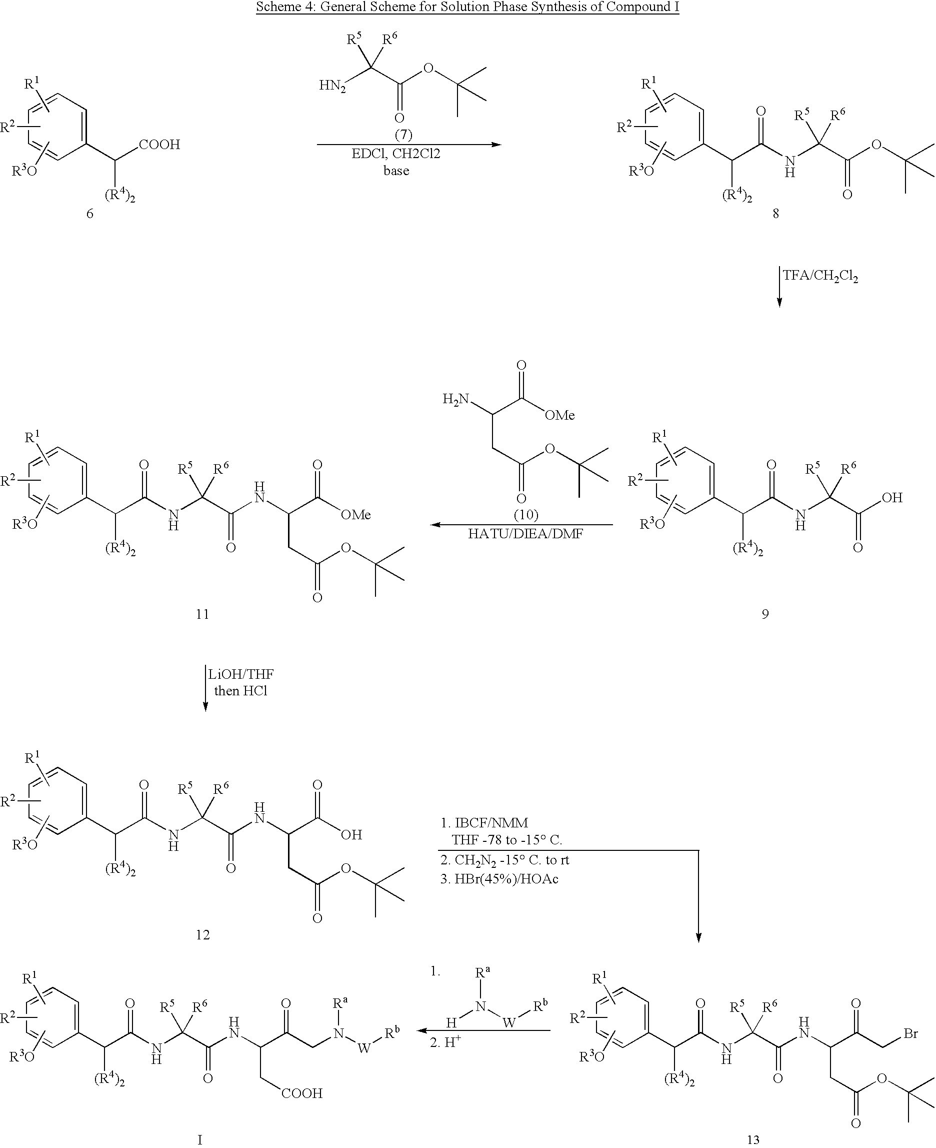

The solution phase synthesis of compound I is outlined in Scheme 4. Acid 6 is first reacted with an appropriate amine 7 using EDCI as the coupling reagent to give amide 8. The t-butyl ester in 8 is cleaved with trifluoroacetic acid to yield carboxylic acid 9, which is further reacted with β-t-butyl aspartic acid methyl ester (10) in the presence of HATU and diisopropylethylamine, giving product 11. The methyl ester in 11 is hydrolyzed using LiOH in TBF and acid 12 thus obtained is reacted with iso-butyl chloroformate in the presence of N-methylmorpholine. The mixed anhydride thus generated is reacted with diazomethane in situ. The mixture is then treated with a solution of 45% HBr in glacial acetic acid (1:1, v/v) to afford bromomethyl ketone 13. Reaction of 13 with a suitable amine HNR^{a}(WR^{b}) followed by cleavage of the t-butyl ester with an acid furnish the final product 1.

The invention is further illustrated using the following non-limiting examples.

### EXAMPLE 1

### (3S)-3-[((2S)-2-{[2-(5-acetyl-2-methoxyphenyl)acetyl]amino}-3-methylbutanoyl)amino]-5-[(2-chloro-6-fluorobenzoyl)amino]-4-oxopentanoic acid

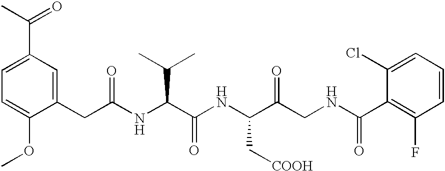

### Step 1: Preparation of Resin A

A suspension of amino-Merrified resin (Novabiochem, 30 grams, 31.2 mmol), acid 5 (14.7 g, 46.8 mmol), EDCI (10.77 g, 56.12 mmol) and HOBT (8.6 g, 56.16 mmol) in DMF (240 mL) was shaken on a orbital shaker at 190 rpm overnight. The mixture was filtered and the residual resin washed sequentially with DMF, methanol, dichloromethane and methanol and dried under vacuum. The resin was suspended in a solution of TFA/dichloromethane (1:2, 300 mL) and shaken for 2 hours on an orbital shaker. The suspension was filtered, washed with dichloromethane (5×) and methanol (5×) and then dried under vacuum overnight to yield Resin A (40.5 g, 0.81mmol/g).

### Step 2: t-Butyl (3S)-5-bromo-3-[(9H-9-fluorenylmethoxy)carbonyl]amino-4-oxo-pentanoate (1)

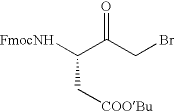

To a solution of N-Fmoc-L-aspartic acid β-t-butyl ester (21.0 g, 51.0 mol) in 300 mL of tetrahydrofuran (THE) at −78° C. was added N-methylmorpholine (NMM, 7.9 mL, 71.4 mmol) followed by iso-butyl chloroformate (IBCF, 8.6 mL, 66.3 mmol). After stirring for 30 minutes at −78° C., this mixture was warmed to −15° C. for 15 minutes. To the mixture was then added excess amount of diazomethane in ether (1 M) with stirring until a yellow color persisted at room temperature. The solution was then stirred at room temperature for 30 minutes, recooled back to 0° C. and treated with a solution of HBr(45% aqueous)/AcOH (1/1, v/v, 100 mL) for 5 minutes, and diluted with ethyl acetate and water. The organic phase was separated, washed with water and brine, dried over magnesium sulfate, filtered and concentrated. The crude product was purified by flash chromatography. Eluting with hexanes/ethyl acetate (3:1) afforded the desired product as a white powder (20 g, 81% yield). ¹H NMR (400 MHz, acetone-d₆): δ7.85 (d, 2H), 7.69 (d, 2H), 7.41 (t, 2H), 7.32 (t, 2H), 7.02 (bd, 1H, NH), 4.70 (dd, 1H), 4.51-4.41 (m, 2H), 4.38-4.30 (2×d, 2H), 4.25 (t, 1H), 2.25 (dd, 1H), 2.70 (dd, 1H), 1.41 (s, 9H).

### Step 3: t-Butyl (3S)-5-azido-3-[(9H-9-fluorenylmethoxy)carbonyl]amino-4-oxo-pentanoate (2)

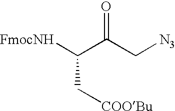

To a solution of bromide 1 (4.88 g, 10 mmol) in DMF (100 mL) was added NaN₃ (1.3 g, 20 mmol) and the mixture was stirred under N₂ for 2 h and cooled to 0° C. The mixture was then extracted with ether and the ether layer was washed with water and brine. After drying over MgSO₄, the organic solution was filter and concentrated. The residue was purified by column chromatography. Eluting with ethyl acetate/hexanes (1/5 v) afforded the desired azide 2 (4.1 g). ¹H NMR (400 MHz, acetone-d₆) δ7.86 (d, 2H), 7.68 (d, 2H), 7.41 (t, 2H), 7.32 (t, 2H), 7.01 (br d, 1H, NH), 4.574.43 (m, 3H), 4.30-4.22 (m, 3H), 2.85-2.81 (m, 1H), 2.71 (dd, 1H), 1.41 (s, 9H).

### Step 4: preparation of (5-acetyl-2-methoxyphenyl)acetic acid (14)

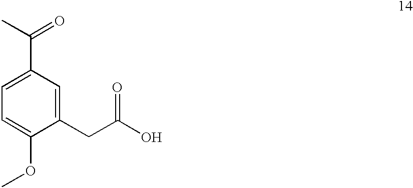

A mixture of 5-acetyl-2-methoxybenzyl nitrite in acetic acid (40 mL), concentrated H2SO₄ (40 mL) and H₂O (40 mL) was heated to 120° C. for 4 hours and cooled to room temperature. The mixture was diluted with water and extracted with ethyl acetate (3×). The organic extracts were combined, washed with water, brine, dried over MgSO₄ and concentrated. The crude product was recrystallized from ethyl acetate/hexanes to give the desired product 14 as a white powder. ¹H NMR (400 MHz, acetone-d₆) δ7.95 (dd, 1H), 7.90 (d, 1H), 7.10 (d, 1H), 3.91 (s, 3H), 3.68 (s, 2H), 2.53 (s, 3H).

### Step 5: loading of ketone 2 to Resin A and preparation of Resin F

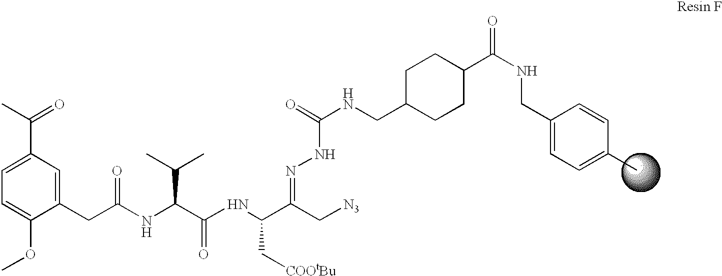

To a suspension of Resin A (4 g, 0.7 mmol/g) in THF (40 mL) in a fritted reservoir was added azide 2 (1.79 g) and acetic acid (0.09 mL) and the mixture was agitated overnight and filtered. The resin was washed thoroughly with dichloromethane, ethyl acetate and ether and then dried under high vacuum to afford 4.9 g of loaded resin (0.58 mmol/g).

The above resin was treated with 20% (v) piperidine in DMF (26 mL) for 20 minutes and filtered. The residue resin was washed thoroughly with DMF, methanol, dichloromethane, ethyl acetate and ether and dried. To the dried resin suspended in DMF (35 mL) was added Fmoc-Valine-OH (1.63 g), HATU (1.82 g) and DIEA (0.84 mL) and the suspension was mixed for 4 hours at room temperature and the filtered. After washing twice with DMF, the resin was treated with 20% (v) piperidine in DMF (26 mL) for 20 minutes and washed and dried as described above. A portion of the dried resin (2.8 g) was suspended in DMF (25 mL) and to it was added acid 14 (0.7 g), HATU (1.28 g) and DIEA (0.58 mL) and the mixture was agitated for 3 hours and washed with DMF (10×). To the washed resin was added a solution of dithiothreitol (2 mL, 2M in DMF) and DIEA (2 ML, 1M in DME) and the suspension was heated to 60° C. under N₂ for 35 min and cooled to r.t. The resin was then filtered and washed thoroughly with DMF, MeOH, THF, ethyl acetate and ether. The washed resin was dried under high vacuum overnight to afford Resin F.

### Step 6: title compound

To a suspension of Resin F (60 mg) in DMF (1 mL) was added 2-chloro-6-fluorobenzoic acid (31 mg), HATU (68 mg) and DIEA (31 μL) and the mixture was agitated at r.t. for 2 h. The resin was washed thoroughly with DMF, ethyl acetate, TBF, ethyl acetate and ether and then treated with TFA/H₂O (1 IL, 9/1, v/v) for 1 h. The mixture was filtered and the residue washed with dichloromethane and acetonitrile. The filtrate and wash solutions were combined and concentrated. The residue was triturated with ether to give the title compound as a white powder. ¹H NMR (400 MHz, acetone-d₆/CD₃OD) δ7.92-7.88 (m, 2H), 7.42 (mn, 1H), 7.27 (d, 1H), 7.18 (t, 1H), 7.05 (d, 1H), 4.78 (mn, 1H), 4.50-4.40 (m, 2H), 4.32 (m, 1H), 3.90 (s, 3H), 3.69-3.61 (2×d, AB, 2H), 2.91 (dd, 1H), 2.80 (dd, 1H), 2.49 (s, 3H), 2.12 (m, 1H), 0.90 (2×d, 6H). MS (−APCI): m/z 590.2 (M−1)⁻.

Alternatively, reaction of Resin F with an appropriate acid chloride or an suitable chloroformate in dichloromethane in the presence of DIEA followed by cleavage with TFA/H₂O also gave the desired product in good purity.

Examples 2-20 were prepared accordingly.

### EXAMPLE 21

### (3S)-3-[((2S)-2-{[2-(5-acetyl-2-methoxyphenyl)acetyl]amino}-3-methylbutanoyl)amino]-5-[(2-fluorobenzenesulfonyl)amino]-4-oxopentanoic acid

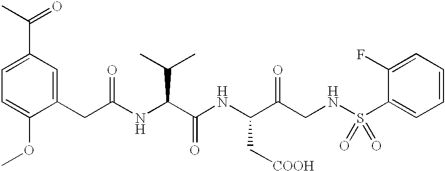

To a suspension of Resin F (0.29 g) in dichloromethane (3 mL) was added 2-fluorophenylsulfonyl chloride (0.175 g) and diisopropylethylamine (0.165 mL) and the suspension was agitated for 2 hours at room temperature. The resin was filtered and washed thoroughly with dichloromethane, ethyl acetate and ether and then treated with a solution of TFA/H₂O (9:1 v/v, 5 mL) for 1 hour and filtered. The resin was washed with dichloromethane and CH₃CN. The filtrate and washing solutions were combined, concentrated in vacuo and trituated with ether to give a white solid (58 mg). ¹H NMR (400 MHz, CD₃OD) δ7.99 (m, 1H), 7.89 (m, 1H), 7.81 (m, 1H), 7.60 (m, 1H), 7.35-7.25 (m, 2H), 7.06 (d, 1H), 4.60 (t, 1H), 4.15-4.03 (m, 3H), 3.90 (s, 3H), 3.70-3.55 (2×d, AB, 2H), 2.83 (dd, 1H), 2.67 (dd, 1H), 2.53 (s, 3H), 2.03 (m, 1H), 0.90 (m, 6H). MS (−APCI): m/z 592.5 (M−1)⁻.

### EXAMPLE 24

### (3S)-3-[((2S)-2-{[2-(5-acetyl-2-methoxyphenyl)acetyl]amino}-3-methylbutanoyl)amino]-5-[(benzooxazol-2-yl)amino]-4-oxopentanoic acid

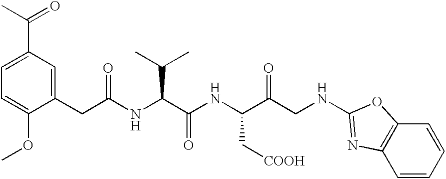

To a suspension of Resin F (0.1 g) in DMF (1.5 mL) was added 2-chlorobenzoxazole (0.04 mL) and diisopropylethylamine (0.03 mL) and the mixture was heated to 80° C. for 4 hours and cooled to room temperature. The mixture was filtered and the resin washed with DMF, dichloromethane, ethyl acetate and ether. The washed resin was treated with a solution of TFA/H₂O (9:1 v/v, 1.5 mL) for 1 hour and filtered. The filtrated was concentrated and trituated with ether to give the desired product (16 mg). ¹H NMR (400 MHz, acetone-d₆/CD₃OD) δ7.83-7.80 (m, 2H), 7.33 (d, 1H), 7.23-7.15 (m, 2H), 7.10 (t, 1H), 7.18 (t, 1H), 6.99 (d, 1H), 4.75 (t, 1H), 4.54 (d, 2H), 4.40 (d, 1H), 4.21 (m, 1H), 3.86 (s, 3H), 3.69-3.57 (2×d, AB, 2H), 2.92 (dd, 1H), 2.76 (dd, 1H), 2.43 (s, 3H), 2.12 (m, 1H), 0.90 (2×d, 6H). MS (−APCI): m/z 551.4 (M−1)⁻.

Example 23 was prepared accordingly.

### EXAMPLE 26

### (3R,3S)-3-{[(2S)-2-({[2-methoxy-5-(3-methyl-1,2,4-oxadiazol-5-yl)-phenyl]acetyl}amino}-3-methylbutanoyl]amino}-4-oxo-5-(pyrrolidin-1-yl)pentanoic acid

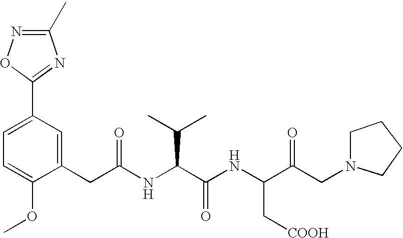

The preparation of this compound is illustrated in Scheme 5.

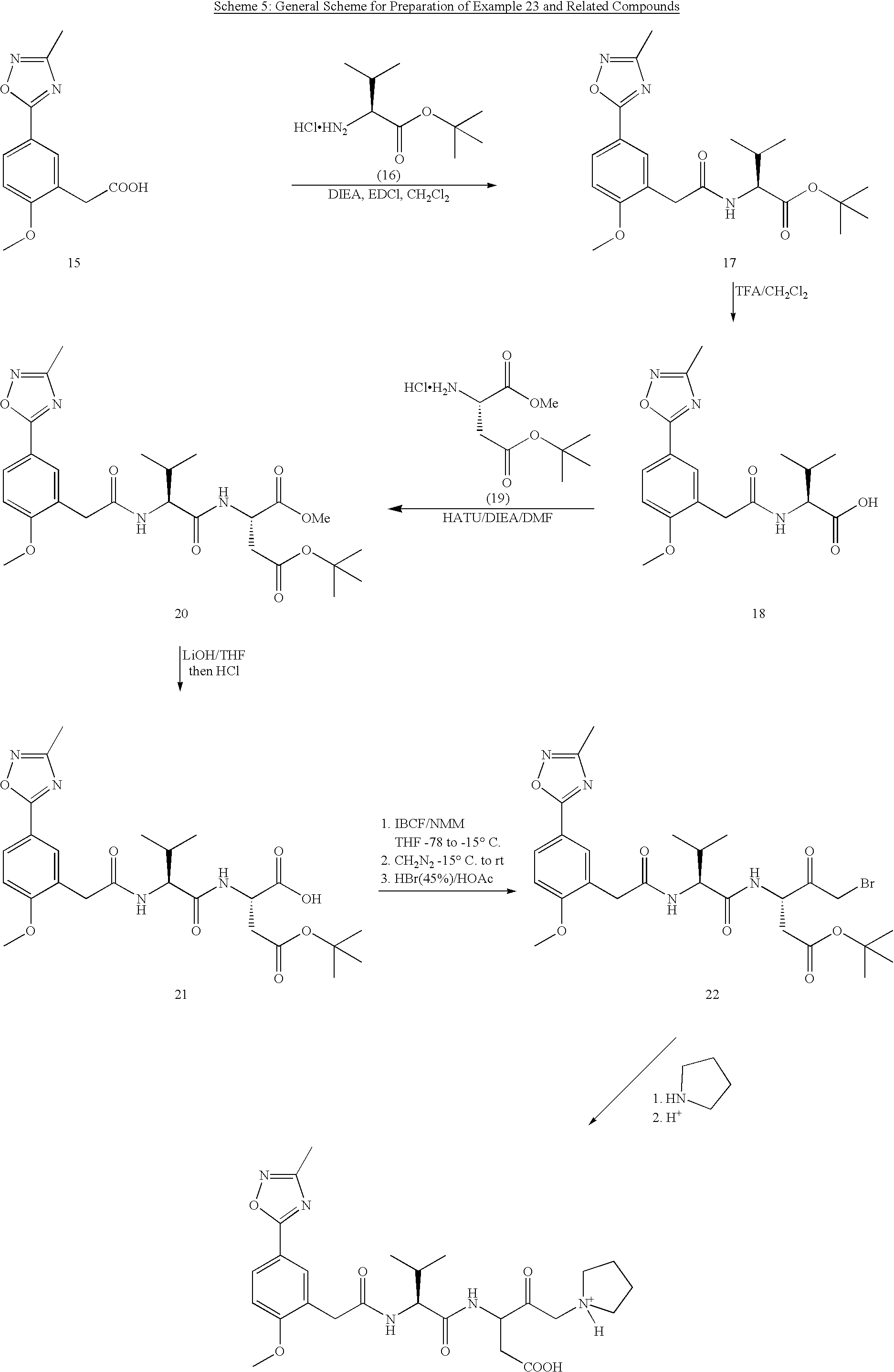

### Step 1: methyl (5-iodo-2-methoxyphenyl)acetate (23)

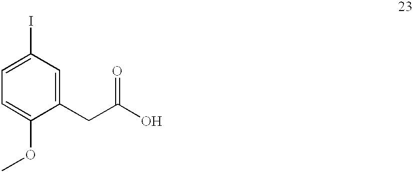

To a solution of 2-methoxyphenylacetic acid (14 g, 84 mmol) in dioxane (100 mL) at 0° C. was added ICl (14 g, 86 mmol) in dioxane (50 mL) over a period of 15 min. The mixture was stirred at 0° C. for an additional 15 min and poured to a mixture of water (2 L) and 5% Na₂S₂O₃ (50 mL). After the solution became clear, the solid was collected by vacuum filtration and washed with water. Drying under vacuum afforded 10 g of 5-iodo-2-methoxyphenylaceitc acid. ¹H NMR (400 MHz, acetone-d₆): δ7.55 (d, 1H), 7.54 (s, 1H), 6.80 (d, 1H), 3.80 (s, 3H), 3.58 (s, 2H).

The acid obtained above was added to a solution of acetyl chloride (50 mL) in methanol (500 mL) and the mixture was stirred overnight and then heated to reflux for 2 h. After cooling to room temperature, the mixture was concentrated and the crude product was purified by flash column chromatography. Eluting with EtOAc/Hexanes (1/9) furnished desired product 23 (9 g). ¹H NMR (400 MHz, acetone-d₆): δ7.58 (d, 1H), 7.55 (s, 1H), 6.82 (d, 1H), 3.80 (s, 3H), 3.60 (s, 3H), 3.58 (s, 2H).

### Step 2: preparation of [2-methoxy-5-(3-methyl-1.2,4-oxadiazol-5-yl)phenyl]acetic acid (15) and the title compound

The following reaction was carried out according to the literature procedure (see: Young, J. R. and DeVita R. J., Tetrahedron Lett. 39, 3931 (1998)).

A mixture containing iodide 23 (700 mg, 2.3 mmol), (PPh₃)2PdCl₂ (322 mg, 0.46 mmol), methylamidoxime (518 mg, 6.9 mmol) and triethylamine (644 mL, 4.6 mmol) in toluene (10 mL) was carefully purged with CO and then heated to 90° C. for 10 h and cooled to room temperature. Concentration of the volatiles gave the crude product which was purified by column chromatography. Eluting with EtOAc/hexanes (1:4) gave the desired product as a white solid. ¹H NMR (400 MHz, acetone-d₆): δ8.05 (d, 1H), 8.01 (s, 1H), 7.20 (d, 1H), 3.92 (s, 3H), 3.75 (s, 2H), 3.65 (s, 3H), 2.37 (s, 3H). The ester (900 mg) was dissolved in a solution of THF (10 mL), methanol (10 mL) and water (10 mL). To the solution was added LiOH (5 mL, 1M in water) and the mixture was stirred at room temperature for 4 hours, acidified with 1N HCl and extracted with ethyl acetate (3×). The extracts were combined, washed with water and brine, dried over MgSO₄ and concentrated to afford the desired acid as a white powder. ¹H NMR (300 MHz, acetone-d₆): δ8.05 (d, 1H), 8.01 (s, 1H), 7.18 (d, 1H), 3.94 (s, 3H), 3.71 (s, 2H), 2.37 (s, 3H).

A mixture of acid 15 (2.48 g, 10 mmol), (S)-valine t-butyl ester hydrochloride (16) (2.3 g), EDCI (2.3 g) and diisopropylethylamine (5.3 mL) in dichloromethane (100 mL) was stirred at room temperature for 2 hours. Most of solvents were removed in vacuo and the residue was diluted with 1 N HCl and ether. The layers were separated and the aqueous layer was extracted twice with ether. The organic layers were combined, washed with 1 N HCl, water and aqueous sodium bicarbonate. After drying over MgSO₄ and vacuum filtration, the solution was concentrated in vacuo to afford the desired product 17 as a white powder (3.7 g). ¹H NMR (300 MHz, acetone-d₆): δ8.01 (m, 2H), 7.20 (d, 1H), 7.10 (br d, 1H), 4.30 (dd, 1H), 3.96 (s, 3H), 3.65 (AB dd, 2H), 2.36 (s, 3H), 2.12 (m, 1H), 1.41 (s, 9H) and 0.90 (2×d, 6H).

Product 17 from above was treated with a solution of 20% TFA (v) in dichloromethane for 1 hour at room temperature. Concentration in vacuo yielded the desired acid 18 as a white powder. ¹H NMR (400 MHz, acetone-d₆): δ8.00 (m, 2H), 7.22 (br d, 1H), 7.19 (d, 1H), 4.44 (dd, 1H), 3.94 (s, 3H), 3.70 (AB dd, 2H), 2.37 (s, 3H), 2.15 (m, 1H), 0.92 (2×d, 6H).

To a solution of Acid 18 (2.1 g, 6.05 mmol) in DMF (30 mL) was added (S)-β-t-butyl aspartic acid methyl ester hydrochloride (19) (1.6 g, 6.66 mmol), HATU (2.53 g, 6.66 mmol) and diisopropylethylamine (2.41 mL) and the solution was stirred at room temperature for 2 hours and diluted with water and ether. The layers were separated and aqueous layer extracted twice with ether. The organic layers were combined, washed with 1 N HCl, water and brine, and dried over Na₂SO₄. Evaporation of solvents in vacuo gave a white solid which was recrystallized from ethyl acetate and hexanes. The product 20 (3 g) thus obtained was a white powdery solid. ¹H NMR (400 Mz, acetone-d₆): δ8.02-7.98 (m, 2H), 7.61 (br d, 1H), 7.20 (d, 1H), 7.12 (br d, 1H), 4.74 (m, 1H), 4.33 (dd, 1H), 3.96 (s, 3H), 3.73-3.60 (m, 5H), 2.80-2.68 (m, 2H), 2.36 (s, 3H), 2.10 (m, 1H), 1.38 (s, 9H), and 0.91-0.87 (m, 6H).

The methyl ester in 20 was hydrolyzed as follow: To a solution of 20 (2.7 g) in THF (56 mL) was added LiOH (5.6 mL, 1 M) at room temperature and the mixture was stirred for 1 hour and acidified with 1 N HCl. The mixture was then extracted with ethyl acetate (3×) and the extracts washed with water and brine, dried over Na₂SO₄ and concentrated to give acid 21 as a white powder (2.5 g). ¹H NMR (500 MHz, acetone-d₆): δ8.03-7.99 (m, 2H) 7.95 (br d, 1H), 7.50 (br d, 1H), 7.20 (d, 1H), 4.69 (dd, 1H), 4.34 (dd, 1H), 3.95 (s, 3H), 3.70 (d, 1H), 3.61 (d, 1H), 2.76 (dd, 1H), 2.67 (dd, 1H), 2.38 (s, 3H), 2.10 (m, 1H), 1.40 (s, 9H), 0.90 (mn, 6H).

To a solution of acid 21 (2.5 g, 4.83 mmol) in TBF (250 mL) at −78° C. under a nitrogen atmosphere was added N-methylmorpholine (0.69 mL) and iso-butyl chloroformate (0.75 mL). The mixture was stirred at the temperature for 30 min and allowed to warm to −15° C. for 30 min. Excess amounts of diazomethane in ether was added (until the solution remained yellow at room temperature) and the resultant mixture was stirred at room temperature for 30 min and cooled to 0° C. To it was added a solution of 1:1 (v/v) 45% HBr and glacial acetic acid (40 mL) and the mixture was diluted with water and extracted with ethyl acetate (3 ×). The extracts were combined, washed with water and brine, dried over MgSO₄ and filtered. The filtrate was concentrated in vacuo and co-evaporated with toluene (2×) to give a white solid. The solid was recrystallized from ethyl acetate and hexanes to give bromomethyl ketone 22 as a white solid (2.8 g from two crops). ¹H NM (500 MHz, acetone-d₆): δ8.03-7.97 (m, 3H), 7.28 (br d, 1H), 7.20 (d, 1H), 4.80 (dd, 1H), 4.40 (d, 1H), 4.28-4.12 (m, 2H), 3.96 (s, 3H), 3.72-3.62 (dd, 2H), 2.87 (dd, 1H), 2.70 (dd, 1H), 2.37 (s, 3H), 2.13 (m, 1H), 1.40 (s, 9H), 0.92 (m, 6H).

The title compound: To a solution of bromomethyl ketone 22 (1.01 g) in THF (40 mL) was added pyrrolidine (0.28 mL) and the resulting mixture was stirred at room temperature for 1 hour and diluted with water and aqueous sodium bicarbonate. The mixture was then extracted with ethyl acetate (3×) and the extracts washed with brine. After drying over MgSO₄, the solution was filtered and concentrated. The residue was purified by column chromatography to yield the desired product as an inseparable mixture of two diastereomers (0.9 g). ¹H NM (500 MHz, acetone-d₆): δ8.02 (d, 1H), 8.00 (s, 1H), 7.75-7.65 (2×d, 1H, the NH for the two isomers), 7.20-7.14 (m, 2H), 4.82-4.70 (2×dd, 1H), 4.30 (m, 1H), 3.96 (s, 3H), 3.70-3.25 (m, 4H), 2.85-2.65 (m, 2H), 2.49 (m, 4H), 2.35 (s, 3H), 2.10 (m, 1H), 1.70 (m, 4H), 1.40 (2×s, 9H) and 0.90 (m, 6H). This compound was then treated with TFA (5 mL) in dichloromethane (20 mL) at room temperature for 3 hour and then concentrated to give the title compound as a gray solid. MS (+ESI): 531.2 (M+1)⁺.

Examples 25, 27-62 were prepared accordingly.

### Assays for Determining Biological Activity

1. Measurement of Caspase Activity by Cleavage of a Fluorogenic Substrate

A fluorogenic derivative of the tetrapeptide recognized by caspase-3 and corresponding to the P₁ to P₄ amino acids of the PARP cleavage site, Ac-DEVD-AMC (AMC, amino-4-methylcoumarin) was prepared as follows: i) synthesis of N-Ac-Asp(OBn)-Glu(OBn)-Val-CO₂H, ii) coupling with Asp(OBn)-7-amino-4-methylcoumarin, iii) removal of benzyl groups.

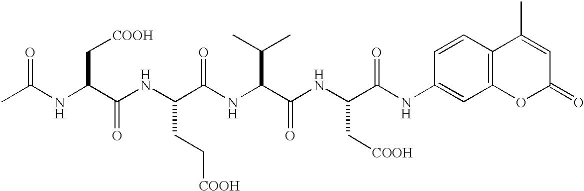

Standard reaction mixtures (300 μL final volume), contained Ac-DEVD-AMC and purified or crude caspase-3 enzyme in 50 mM Hepes/KOH (pH 7.0), 10% (v/v) glycerol, 0.1% (w/v) CHAPS, 2 mM EDTA, 5 mM dithiothreitol, and were incubated at 25° C. Reactions were monitored continuously in a spectrofluorometer at an excitation wavelength of 380 nm and an emission wavelength of 460 nm.

2. Cell Death Detection ELISA (Whole Cell Assay)

Photometric immunoassay for the qualitative and quantitative in vitro determination of cytoplasmic histone-associated-DNA-fragments (mono- and oligonucleosomes) after induced cell death. This assay was performed using the commercially available kit from Boehringer Mannheim, cat. No. 1 920 685.

3. In Vivo Myocardial Ischemia and Reperfusion Injury in Rats

Male Sprague-Dawley rats (300-400 g) were fasted overnight, and then anesthetized with intraperitoneal administration of sodium pentobarbital (65 mg/kg). To monitor heart rate and aortic pressure the left carotid artery was isolated and a cannula placed in the vessel. The aortic cannula was interfaced with a pressure transducer which was connected to a physiologic recorder. The left jugular vein was isolated and cannulated for administration of a caspase inhibitor compound or vehicle (2% dimethylsulfoxide in 0.9% NaCl). A left thoracotomy was performed in the region overlying the heart and the pericardium opened, exposing the heart. The origin of the left coronary artery was visualized and a 4.0 suture passed under the artery approximately 2-3 mm from its origin. The ends of the suture were passed through a short length of 2 mm id tubing and coronary artery occlusion effected by placing tension on the suture such that the tube compressed the artery. After initial placement of the suture/occluder, the thoracotomy was closed with a small clamp and opened only to effect occlusion and reperfusion of the artery. A Lead II electrocardiograph (ECG) signal was obtained by placing subdermal platinum leads and continuously monitored. After a baseline period of 20-30 minutes the left coronary artery was occluded for 45 minutes. The period of reperfusion was 3 hours. The caspase inhibitor or vehicle was administered as a first bolus 5 minutes before the onset of ischemia and a second bolus was administered again at the onset of reperfusion. Additionally, an infusion was initiated immediately after the first bolus dose. Control animals received the vehicle alone in equal volumes to the caspase inhibitor treated animals. At the end of reperfusion the animals were euthanized and infarct size determined using a dual staining technique (1.5% w/v triphenyltetrazolium chloride to demarcate infarct tissue and 0.25% w/v Evan's blue to demarcate the area at risk of infarct. The heart was subsequently cut transversely into 4 slices of equal thickness, and infarct size and area at risk quantified using planimetry.

Using the above procedure, it is demonstrated that administration of a caspase inhibitor reduces infarct size in the rat subjected to 45 minutes of regional ischemia and 3 hours of reperfusion.

4. in vivo Rat Middle Cerebral Artery Occlusion (MCAO)

Male Wistar rats are anesthetized with isoflurane (1.5%-3%) using a face mask for surgical isolation of the right middle cerebral artery (MCA) and the right and left common carotid artery. Anesthetized animals are then placed on a water jacketed heating pad to maintain normal body temperature. To ensure adequate hydration throughout the experiment, rats are administered 10-15 ml/kg of sterile 0.9% NaCl subcutaneously after anesthesia. The rats are then placed on its right side and the heads immobilized. An incision is made directly in front of the ear, extending down from the base of the ear approximately 1.5 cm. The skin is held back and the salivary gland dissected from surrounding tissues. The gland is pulled forward and down away from surgical field. The temporalis muscle is dissected and retracted. Fascia overlying the skull is removed, leaving a clean section of the skull. The bone of the skull is “thinned” with surgical drill (2 mm burr) and remaining skull dissected away from the dura with forceps. The dura is removed, revealing the MCA. The right MCA is occluded using a 1 mm microclip. The right common carotid artery is permanently occluded using a suture. The left common carotid artery is occluded for a period of time equal to the MCA. Rats are awake within 10 minutes after the end of anesthesia. Analgesis is provided to the rats with oxymorphone (0.1 ml/100 g body weight), once or twice according to veterinary advice.

After surgical isolation of the MCA, the MCA is occluded for a period of 30-120 minutes. The left common carotid artery is occluded for the same period of time as the MCA. In these experiments, compounds are administered by different route (icv, iv or ip), as a bolus and/or continuous infusion, before or after the occlusion. Both the MCA and the left common carotid artery are then reperfused. Animals are then administered prophylactic analgesia, and returned to individual cages. At the end of reperfusion, the animals are euthanized and the brains are cut into 2 mm slices and stained with 1.5% w/v triphenyltetrazolium chloride. The infarct size in the brain is determined using a commercially available imaging system.

Using the above procedure, it is demonstrated that administration of a caspase-3 inhibitor reduces infarct size in the cortex regions of the rat brains when the animals are subjected to a 30 to 90 minutes ischemia and 24 hours of reperfusion.

What is claimed is:

1. A compound represented by Formula I: 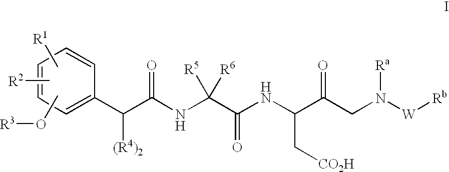
or a pharmaceutically acceptable salt, ester or hydrate thereof, wherein:
W is a bond, —CH₂—, —C(O)— or —S(O)₂—;
R^{a} is selected from R^{b} and H;
R^{b} is independently selected from the group consisting of:
(1) C₁₋₁₀alkyl or C₁₋₁₀alkoxy,
(2) C₃₋₁₁cycloalkyl or a benzofused analog thereof,
(3) phenyl or naphthyl, and
(4) HET¹, wherein HET¹ represents a 5- to 10-membered aromatic, partially aromatic or non-aromatic mono- or bicyclic ring, containing 1-3 heteroatoms selected from O, S and N,
wherein groups (1), (2) and (4) above are optionally substituted with 1-2 oxo groups, and groups (1), (2), (3) and (4) above are further optionally substituted with 1-3 substituents independently selected from the group consisting of:
(a) halo
(b) nitro,
(c) hydroxy,
(d) C₁₋₄alkyl, (e) C₁₋₄alkoxy,
(f) C₁₋₄alkylthio,
(g) C₃₋₆cycloalkyl,
(h) phenyl or naphthyl,
(i) phenoxy, and
(j) a 5 or 6-membered aromatic or non-aromatic monocyclic ring containing 1-3 heteroatoms selected from O, S and N,
wherein groups (d)-(g) above are optionally substituted with oxo and 1-3 substituents independently selected from halo and C₁₋₆alkoxy, and groups (h)-(j) above are optionally substituted with 1-3 substituents independently selected from halo, C₁₋₄alkyl and C₁₋₄alkoxy,
or R^{a} and R^{b} may be joined together with the nitrogen atom to which they are attached to form a 3- to 10-membered non-aromatic monocyclic ring, or a benzofused analog thereof, containing 0-2 additional heteroatoms selected from O, S and N, said ring being optionally substituted with 1-2 oxo groups and further optionally substituted with 1-3 substituents independently selected from the group consisting of:
(g) halo,
(h) C₁₋₄alkyl or C₁₋₄alkoxy, each optionally substituted with 1-3 substituents independently selected from halo and C₁₋₄alkoxy, and
(i) phenyl, naphthyl or benzyl, each optionally substituted with 1-3 substituents independently selected from halo and C₁₋₄alkyl, optionally substituted with halo;
R¹ and R² are independently selected from the group consisting of:
(1) H,
(2) halo,
(3) hydroxy,
(4) nitro,
(5) cyano,
(6) C₁₋₁₀alkyl, C₃₋₁₀cycloalkyl, C₁₋₁₀alkoxy, —S(O)₀₋₂C₁₋₁₀alkyl or —NHC₁₋₁₀alkyl, each optionally substituted with 1-2 oxo groups and further optionally substituted with 1-3 substituents independently selected from the group consisting of:
(a) halo,
(b) hydroxy
(c) cyano,
(d) C₁₋₄alkoxy,
(e) —NHR⁷,
(f) phenyl or naphthyl, and
(g) HET², wherein HET² represents a 5- or 6-membered aromatic or non-aromatic monocyclic ring containing 1-4 heteroatoms selected from O, S and N, said HET² being optionally substituted with oxo and further optionally substituted with 1-2 substituents independently selected from halo and C₁₋₄alkyl, said C₁₋₄alkyl being optionally substituted with 1-3 halo groups,
(7) phenyl or naphthyl,
(8) phenoxy or —S(O)₀₋₂phenyl,
(9) —O-HET² or —S-HET², said HET² being optionally substituted with oxo and further optionally substituted as defined below, and
(10) HET³, wherein HET³ is a 5- or 6-membered aromatic or non-aromatic monocyclic ring, or a benzofused analog thereof, containing 1 to 4 heteroatoms selected from O, S and N, said HET³ being optionally substituted with oxo and further optionally substituted as defined below,
wherein groups (7)-(10) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups,
or if R¹ and R² reside on adjacent atoms then R¹ and R² may be taken in combination with the carbon atoms to which they are attached to form a 5- to 7-membered aromatic or non-aromatic monocyclic ring containing 0-2 heteroatoms selected from O, S and N, said ring optionally substituted with halo and C₁₋₄alkyl, said C₁₋₄alkyl optionally substituted with halo;
R³ is C₁₋₆alkyl, optionally substituted with 1-2 oxo groups and further optionally substituted with 1-3 substituents independently selected from the group consisting of:
(a) halo,
(b) hydroxy
(c) cyano,
(d) C₁₋₄alkoxy,
(e) —NHR⁷,
(f) —S(O)₀₋₂C₁₋₄alkyl, and
(g) HET², optionally substituted with oxo and further optionally substituted with 1-2 substituents independently selected from halo or C₁₋₄alkyl, said C₁₋₄alkyl being optionally substituted with 1-3 halo groups;
each R⁴ is independently selected from the group consisting of: H, halo, hydroxy, C₁₋₆alkyl and C₁₋₄alkoxy, said C₁₋₆alkyl and C₁₋₄alkoxy being optionally substituted with 1-3 halo groups;
R⁵ is selected from the group consisting of: H, phenyl, naphthyl, C₁₋₆alkyl optionally substituted with OR⁸ and 1-3 halo groups, and C₅₋₇ cycloalkyl;
R⁶ represents H;
or R⁵ and R⁶ may be taken in combination with the carbon atom to which they are attached to form a monocyclic ring of 4-7 members, optionally containing one heteroatom selected from O, S and N, said ring optionally substituted with halo and C₁₋₄alkyl;
R⁷ is H or C₁₋₄alkyl, optionally substituted with halo; and
R⁸ is selected from the group consisting of: H, C₁₋₅alkyl optionally substituted with 1-3 halo groups, and benzyl optionally substituted with 1-3 substituents independently selected from halo, C₁₋₄alkyl and C₁₋₄alkoxy.

2. A compound according to claim 1 wherein R¹ is selected from the group consisting of:
(1) halo,
(2) hydroxy,
(3) nitro,
(4) cyano,
(5) C₁₋₁₀alkyl, C₃₋₁₀cycloalkyl, C₁₋₁₀alkoxy, —S(O)₀₋₂C₁₋₁₀alkyl or —NHC₁₋₁₀alkyl, each optionally substituted with 1-2 oxo groups and further optionally substituted with 1-3 substituents independently selected from the group consisting of:
(a) halo,
(b) hydroxy
(c) cyano,
(d) C₁₋₄alkoxy,
(e) —NHR⁷,
(f) phenyl or naphthyl, and
(g) HET², wherein HET² represents a 5- or 6-membered aromatic or non-aromatic monocyclic ring containing 1-4 heteroatoms selected from O, S and N, said HET² being optionally substituted with oxo and further optionally substituted with 1-2 substituents independently selected from halo and C₁₋₄alkyl, said C₁₋₄alkyl being optionally substituted with 1-3 halo groups,
(6) phenyl or naphthyl,
(7) phenoxy and —S(O)₀₋₂phenyl,
(8) —O-HET² or —S-HET², said KET² being optionally substituted with oxo and further optionally substituted as defined below, and
(9) HET³, wherein HET³ is a 5- or 6-membered aromatic or non-aromatic monocyclic ring, or a benzofused analog thereof, containing 1 to 4 heteroatoms selected from O, S and N, said HET³ being optionally substituted with oxo and further optionally substituted as defined below,
wherein groups (6)-(9) above are each optionally substituted with 1-2 substituents independently selected from the group consisting of: halo, cyano, C₁₋₄alkyl and C₁₋₄alkoxy, said C₁₋₄alkyl and C₁₋₄alkoxy being optionally substituted with 1-3 halo groups,
or if R¹ and R² reside on adjacent atoms then R¹ and R² may be taken in combination with the carbon atoms to which they are attached to form a 5- to 7-membered aromatic or non-aromatic monocyclic ring containing 0-2 heteroatoms selected from O, S and N, said ring optionally substituted with halo and C₁₋₄alkyl, said C₁₋₄alkyl optionally substituted with halo.

3. A compound according to claim 1 wherein R³ is methyl, optionally substituted with 1-3 halo groups.

4. A compound according to claim 1 wherein R⁵ is ethyl, isopropyl or cyclopentyl and R⁶ is H.

5. A compound according to claim 1 wherein W is a bond.

6. A compound according to claim 1 wherein W is —CH₂—.

7. A compound according to claim 1 wherein W is —C(O)—.

8. A compound according to claim 1 wherein R^{a} is H or methyl, optionally substituted with 1-3 halo groups.

9. A compound according to claim 8 wherein R^{b} is phenyl or naphthyl, each optionally substituted with 1-3 substituents independently selected from the group consisting of:
(a) halo
(b) nitro,
(c) hydroxy,
(d) C₁₋₄alkyl,
(e) C₁₋₄alkoxy,
(f) C₁₋₄alkylthio, and
(g) C₃₋₆cycloalkyl,
wherein groups (d)-(g) above are optionally substituted with 1-3 substituents independently selected from halo and C₁₋₄alkoxy.

10. A compound according to claim 8 wherein R^{b} is C₁₋₁₀alkyl or C₁₋₁₀alkoxy, each optionally substituted with 1-2 oxo groups and further optionally substituted with 1-3 substituents independently selected from the group consisting of:
(a) halo
(b) nitro,
(c) hydroxy,
(d) C₁₋₄alkoxy,
(e) C₁₋₄alkylthio,
(f) C₃₋₆cycloalkyl,
(g) phenyl or naphthyl, and
(h) phenoxy,
wherein groups (d)-(f) above are optionally substituted 1-3 substituents independently selected from halo and C₁₋₄alkoxy, and groups (g)-(h) above are optionally substituted with 1-3 substituents independently selected from halo, C₁₋₄alkyl and C₁₋₄alkoxy.

11. A compound according to claim 8 wherein R^{b} is C₃₋₁₁cycloalkyl or a benzofused analog thereof, optionally substituted with 1-2 oxo groups and further optionally substituted with 1-3 substituents independently selected from the group consisting of:
(a) halo
(b) nitro,
(c) hydroxy,
(d) C₁₋₄alkyl,
(e) C₁₋₄alkoxy, and
(f) C₁₋₄alkylthio,
wherein groups (d)-(f) above are optionally substituted with 1-3 substituents independently selected from halo and C₁₋₄alkoxy.

12. A compound according to claim 8 wherein R^{a} is HET¹, optionally substituted with 1-2 oxo groups and further optionally substituted with 1-3 substituents independently selected from the group consisting of:
(a) halo
(b) nitro,
(c) hydroxy,
(d) C₁₋₄alkyl,
(e) C₁₋₄alkoxy,
(f) C₁₋₄alkylthio, and
(g) C₃₋₆cycloalkyl,
wherein groups (d)-(g) above are optionally substituted with 1-3 substituents independently selected from halo and C₁₋₄alkoxy.

13. A compound according to claim 12 wherein HET¹ represents a member selected from the group consisting of: pyridine, pyrimidine, pyridazine, pyrazine, furan, thiophene, thiazole, oxazole and isooxazole, or a benzofused or hydrogenated analog thereof, or both, each optionally substituted with 1-2 oxo groups and further optionally substituted with 1-3 substituents independently selected from the group consisting of:
(a) halo
(b) nitro,
(c) hydroxy,
(d) C₁₋₄alkyl,
(e) C₁₋₄alkoxy,
(f) C₁₋₄alkylthio, and
(g) C₃₋₆cycloalkyl,
wherein groups (d)-(g) above are optionally substituted with 1-3 substituents independently selected from halo and C₁₋₄alkoxy, or
HET¹ is

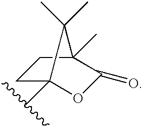

14. A compound according to claim 1 wherein HET² is selected from the group consisting of: butyrolactone, tetrahydrofuran, tetrahydropyran, 2-pyrrolidinone, pyridine and pyrimidine, each optionally substituted with 1-2 substituents independently selected from halo or C₁₋₄alkyl, said C₁₋₄alkyl being optionally substituted with 1-3 halo groups.

15. A compound according to claim 1 wherein HET³ is selected from the group consisting of: 1,2,3-oxadiazole, 1,2,4-oxadiazole, 1,3,4-oxadiazole, 1,2,3-thiadiazole, 1,2,4-thiadiazole, 1,3,4-thiadiazole, thiophene, pyrrole, pyridine, tetrazole, oxazole, thiazole, 1,2,3-triazole, 1,2,4-triazole and 1,3,4-triazole, each optionally substituted with 1-2 substituents independently selected from halo or C₁₋₄alkyl, said C₁₋₄alkyl being optionally substituted with 1-3 halo groups.

16. A compound according to claim 1 wherein R^{a} and R^{b} are joined with the nitrogen atom to which they are attached to form a 3- to 6-membered non-aromatic monocyclic ring, or a benzofused analog thereof, containing 0-2 additional heteroatoms selected from O, S and N, said ring being optionally substituted with 1-2 oxo groups and further optionally substituted with 1-3 substituents independently selected from the group consisting of:
(c) halo,
(d) C₁₋₄alkyl or C₁₋₄alkoxy, each optionally substituted with 1-3 substituents independently selected from halo and C₁₋₄alkoxy, and
(c) phenyl, naphthyl or benzyl, each optionally substituted with 1-3 substituents independently selected from halo and C₁₋₄alkyl, optionally substituted with halo.

17. A compound represented by Formula I: 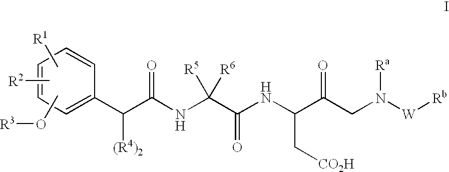
or a pharmaceutically acceptable salt, ester or hydrate thereof, wherein:
W is a bond, —CH₂—, —C(O)— or —S(O)₂—;
R^{a} is H or methyl, optionally substituted with 1-3 halo groups;
R^{b} is independently selected from the group consisting of:
(1) C₁₋₁₀alkyl or C₁₋₁₀alkoxy,
(2) C₃₋₁₁cycloalkyl or a benzofused analog thereof,
(3) phenyl or naphthyl, and
(4) HET¹, wherein HET¹ represents a 5- to 10-membered aromatic, partially aromatic or non-aromatic mono- or bicyclic ring, containing 1-3 heteroatoms selected from O, S and N,
wherein groups (1), (2) and (4) above are optionally substituted with 1-2 oxo groups, and groups (1), (2), (3) and (4) above are further optionally substituted with 1-3 substituents independently selected from the group consisting of:
(a) halo
(b) nitro,
(c) hydroxy,
(d) C₁₋₄alkyl,
(e) C₁₋₄alkoxy,
(f) C₁₋₄alkylthio,
(g) C₃₋₆cycloalkyl,
(h) phenyl or naphthyl,
(i) phenoxy, and
(j) a 5 or 6-membered aromatic or non-aromatic monocyclic ring containing 1-3 heteroatoms selected from O, S and N,
wherein groups (d)-(g) above are optionally substituted with oxo and 1-3 substituents independently selected from halo and C₁₋₄alkoxy, and groups (h)-(j) above are optionally substituted with 1-3 substituents independently selected from halo, C₁₋₄alkyl and C₁₋₄alkoxy,
or R^{a} and R^{b} may be joined with the nitrogen atom to which they are attached to form a 3- to 6-membered non-aromatic monocyclic ring, or a benzofused analog thereof, containing 0-2 additional heteroatoms selected from O, S and N, said ring being optionally substituted with 1-2 oxo groups and further optionally substituted with 1-3 substituents independently selected from the group consisting of:
(d) halo,
(e) C₁₋₄alkyl or C₁₋₄alkoxy, each optionally substituted with 1-3 substituents independently selected from halo and C₁₋₄alkoxy, and
(f) phenyl, naphthyl or benzyl, each optionally substituted with 1-3 substituents independently selected from halo and C₁₋₄alkyl optionally substituted with halo;
R¹ is selected from the group consisting of:
(1) halo,
(2) methoxy,
(3) acetyl, and
(4) 1,2,4-oxadiazol-5-yl, optionally substituted at the 3-position with methyl;
R² is H;
R³ is methyl, optionally substituted with 1-3 halo groups;
each R⁴ is independently selected from the group consisting of: H and hydroxy;
R⁵ is selected from the group consisting of: H, C₁₋₆alkyl optionally substituted with 1-3 halo groups, and C₅₋₇ cycloalkyl; and
R⁶ represents H.

18. A compound according to claim 17 wherein R⁵ is ethyl, isopropyl or cyclopentyl.

19. A compound according to claim 17 wherein:
HET¹ is selected from the group consisting of: pyridine, pyrimidine, pyridazine, pyrazine, furan, thiophene, thiazole, oxazole and isooxazole, or a benzofused or hydrogenated analog thereof, or both, each optionally substituted with 1-2 oxo groups and further optionally substituted with 1-3 substituents independently selected from the group consisting of:
(a) halo
(b) nitro,
(c) hydroxy,
(d) C₁₋₄alkyl,
(e) C₁₋₄alkoxy,
(f) C₁₋₄alkylthio, and
(g) C₃₋₆cycloalkyl,
wherein groups (d) to (g) above are optionally substituted with 1-3 substituents independently selected from halo and C₁₋₄alkoxy, or

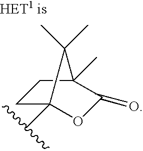

20. A compound selected from the following table:

| Example # | Structure |
|---|---|
| 1 | 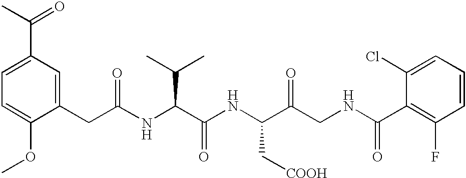 |
| 2 | 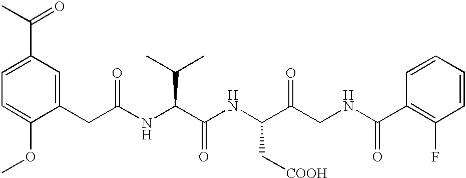 |
| 3 | 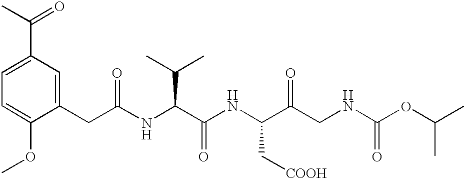 |
| 4 | 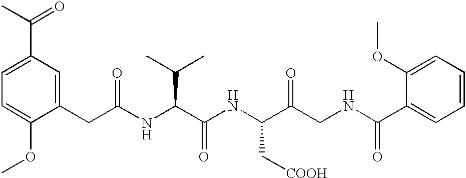 |
| 5 | 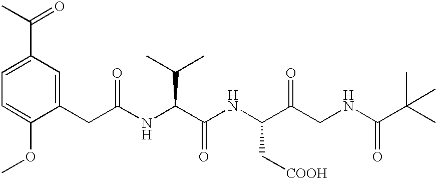 |
| 6 | 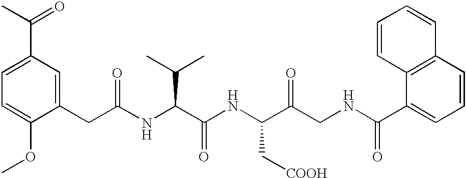 |
| 7 | 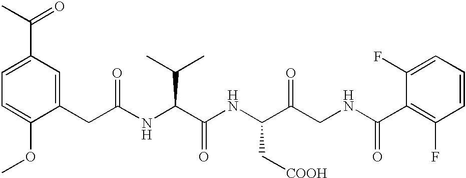 |
| 8 | 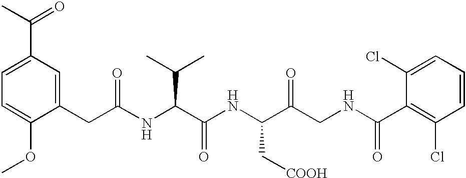 |
| 9 | 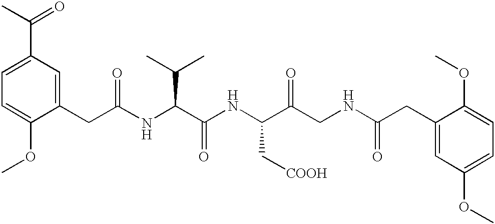 |
| 10 | 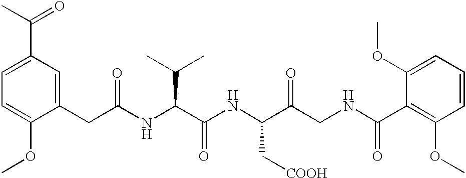 |
| 11 | 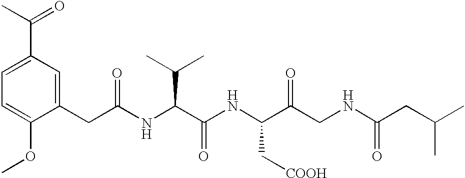 |
| 12 | 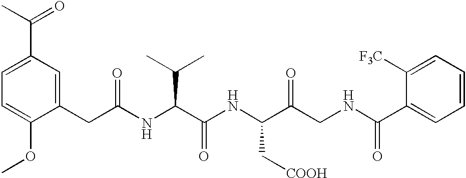 |
| 13 | 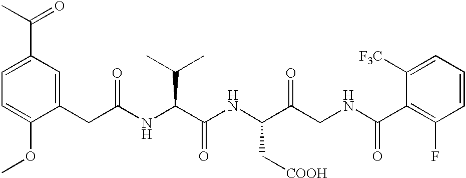 |
| 14 | 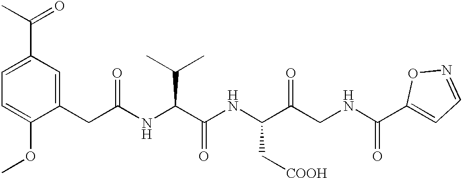 |
| 15 | 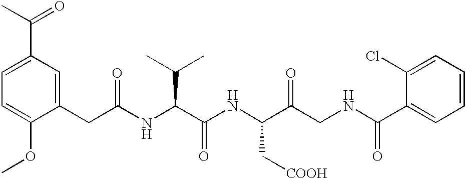 |
| 16 | 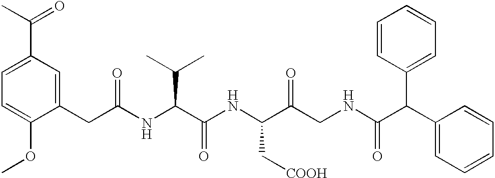 |
| 17 | 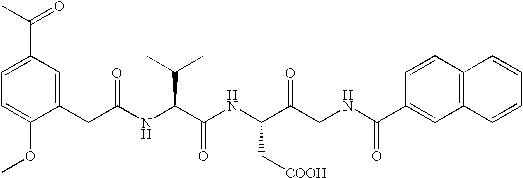 |
| 18 | 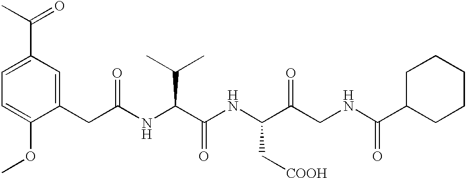 |
| 19 | 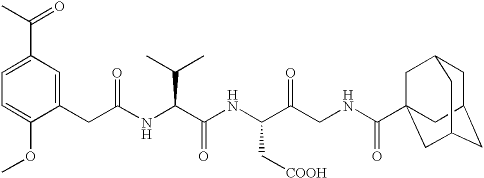 |
| 20 | 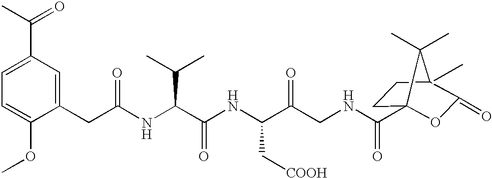 |
| 21 | 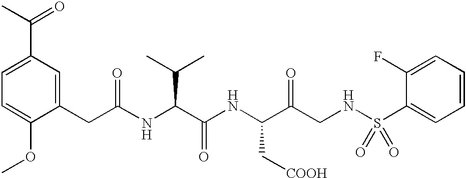 |
| 22 | 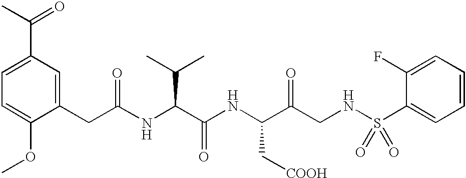 |
| 23 | 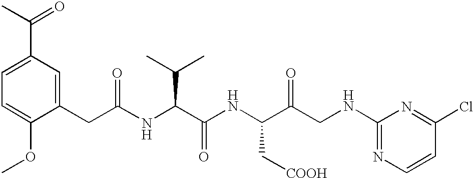 |
| 24 | 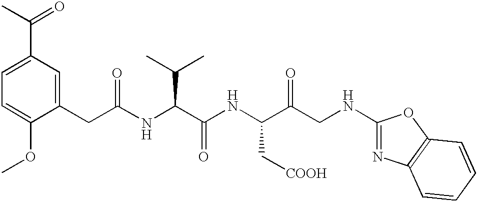 |
| 25 | 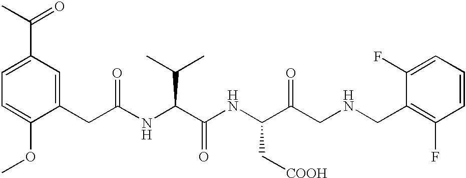 |
| 26 | 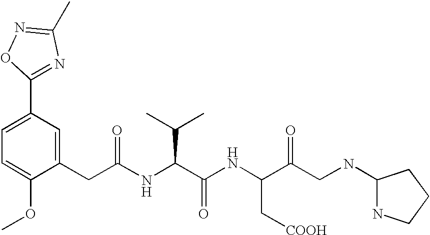 |
| 27 | 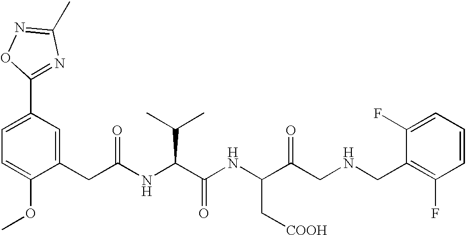 |
| 28 | 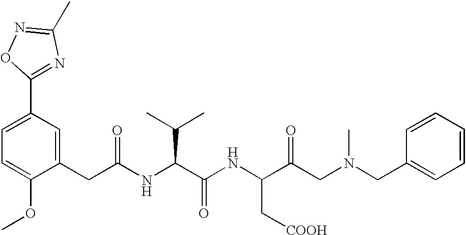 |
| 29 | 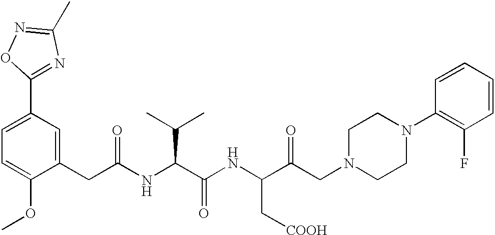 |
| 30 | 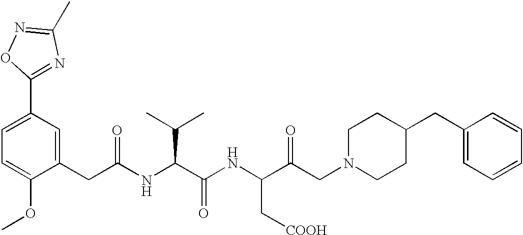 |
| 31 | 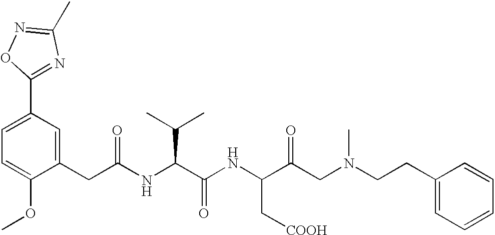 |
| 32 | 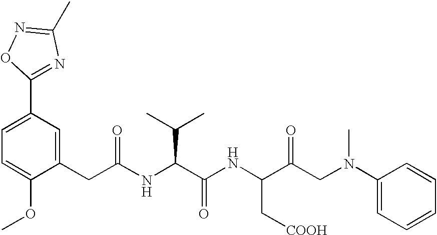 |
| 33 | 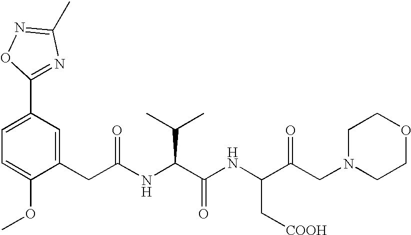 |
| 34 | 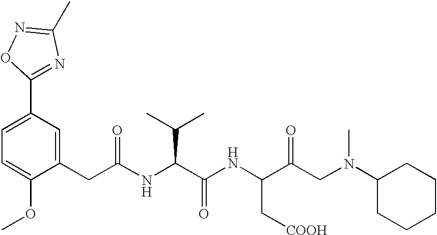 |
| 35 | 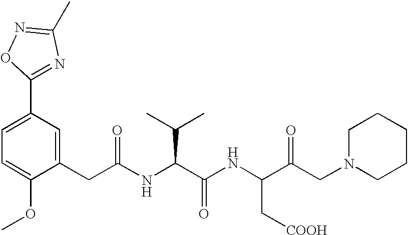 |
| 36 | 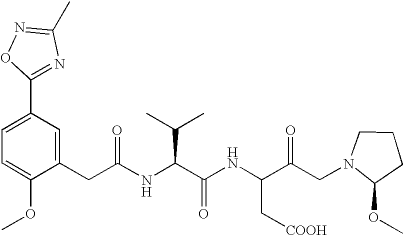 |
| 37 | 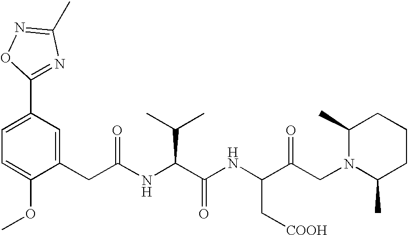 |
| 38 | 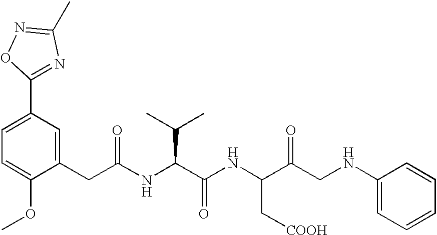 |
| 39 | 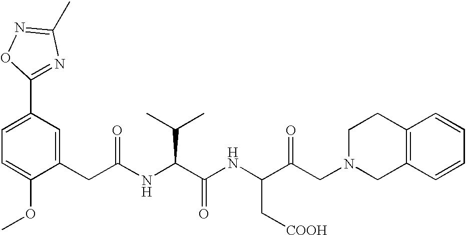 |
| 40 | 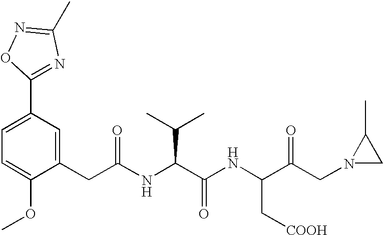 |
| 41 | 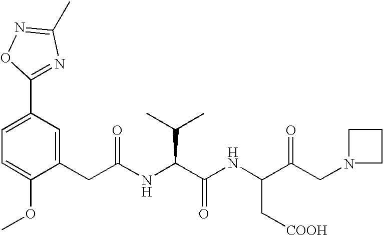 |
| 42 | 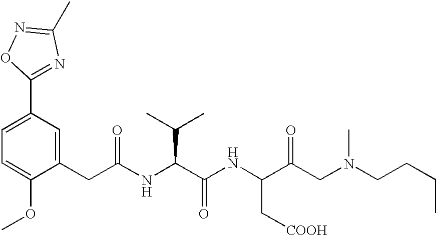 |
| 43 | 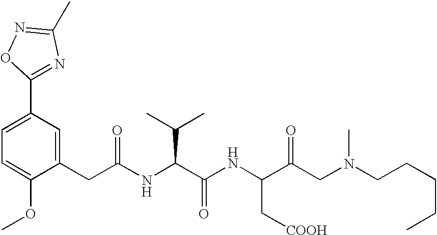 |
| 44 | 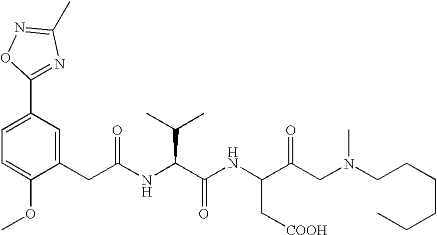 |
| 45 | 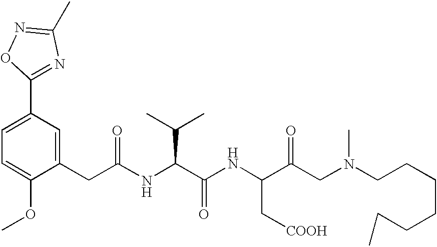 |
| 46 | 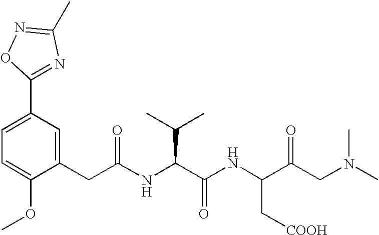 |
| 47 | 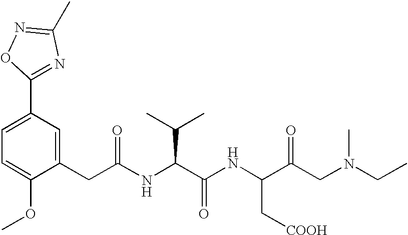 |
| 48 | 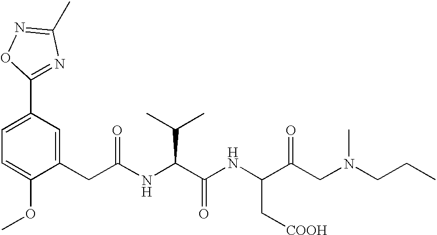 |
| 49 | 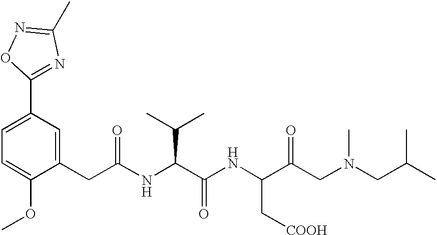 |
| 50 | 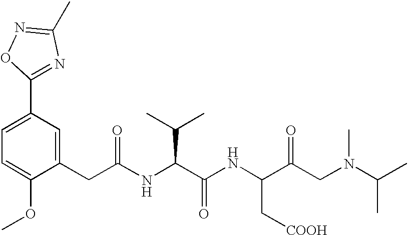 |
| 51 | 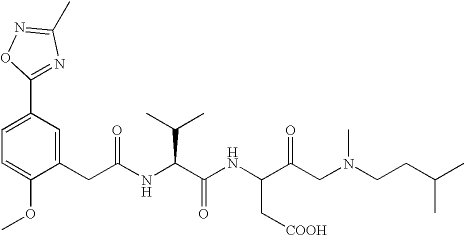 |
| 52 | 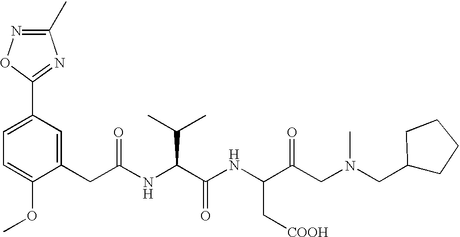 |
| 53 | 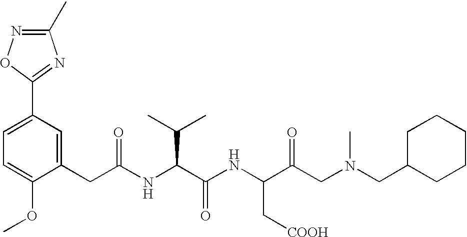 |
| 54 | 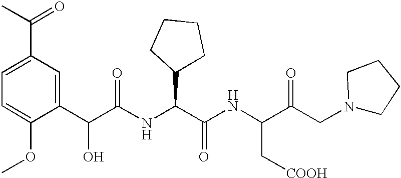 |
| 55 | 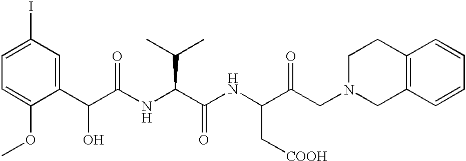 |
| 56 | 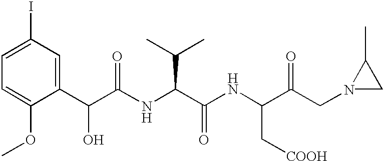 |
| 57 | 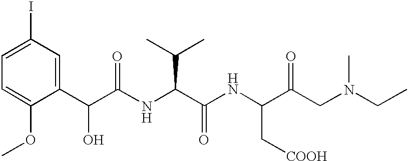 |
| 58 | 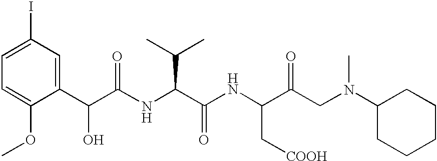 |
| 59 | 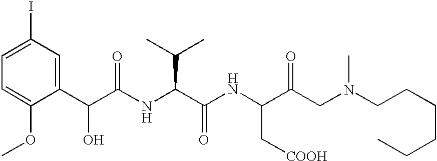 |
| 60 | 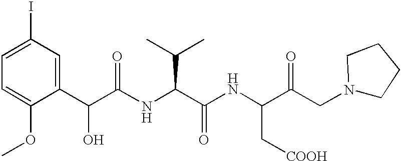 |
| 61 | 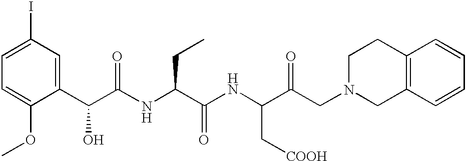 |
| 62 | 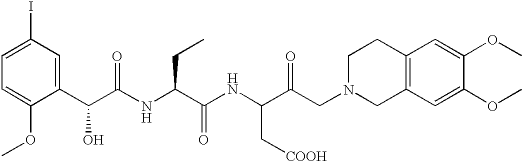 |
| 63 | 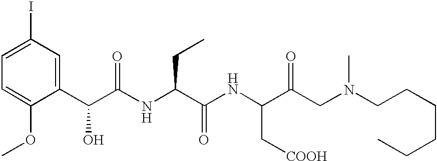 |

or a salt, hydrate, ester, enantiomer or mixture thereof.

21. A pharmaceutical composition comprising a compound in accordance with claim 1 in combination with a pharmaceutically acceptable carrier.

22. A method of treating or preventing a caspase-3 mediated disease or condition in a mammalian patient in need of such treatment or prevention, comprising administering to said patient a compound in accordance with claim 1 in an amount that is effective to treat or prevent said caspase-3 mediated disease.

23. A method in accordance with claim 22 wherein the disease or condition is selected from the group consisting of:
cardiac or cerebral ischemia or reperfusion injury,
type I diabetes,
immune deficiency syndrome or AIDS,
cerebral or spinal cord trauma injury,
organ damage during transplantation,
alopecia,
aging,
Parkinson's disease,
Alzheimer's disease,
Down's syndrome,
spinal muscular atrophy,
multiple sclerosis,
neurodegenerative disorders,
sepsis and
bacterial meningitis.

24. A method in accordance with claim 23 wherein the disease or condition is cardiac and cerebral ischemia or reperfusion injury.

25. A method in accordance with claim 23 wherein the disease or condition is sepsis.

26. A method in accordance with claim 23 wherein the disease or condition is bacterial meningitis.

27. A pharmaceutical composition comprising a compound in accordance with claim 17 in combination with a pharmaceutically acceptable carrier.

28. A method of treating or preventing a caspase-3 mediated disease or condition in a mammalian patient in need of such treatment or prevention, comprising administering to said patient a compound in accordance with claim 17 in an amount that is effective to treat or prevent said caspase-3 mediated disease.
